(19) United States
(12) Patent Application Publication
(10) Pub. No.: US 2021/0093679 A1
(43) Pub. Date: Apr. 1, 2021

(54) ENGINEERED GUT MICROBES AND USES THEREOF

(71) Applicant: NOVOME BIOTECHNOLOGIES, INC., South San Francisco, CA (US)

(72) Inventor: Jonathan Kotula, Berkeley, CA (US)

(21) Appl. No.: 16/967,382

(22) PCT Filed: Feb. 4, 2019
(86) PCT No.: PCT/US2019/016538

§ 371 Date: Aug. 4, 2020

Related U.S. Application Data
(60) Provisional application No. 62/626,586, filed on Feb. 5, 2018.

(51) Int. Cl. A61K 35/744; C12N 9/22; C12N 15/113; C12N 15/70; C12N 15/74

(52) U.S. Cl. CPC A61K 35/744; C12N 9/22; C12N 15/113; C12N 2310/20; C12N 15/74; C12Y 204/01017; C12Y 205/01018; C12N 15/70

(57) ABSTRACT

Methods and compositions for reducing reactivation of detoxified drugs, such as xenobiotic agents with narrow therapeutic indices, are provided. The methods include genetically engineering GI microbes in vivo or in vitro to include modifications that decrease or eliminate the presence of enzymes involved in xenobiotic metabolism, such as β-glucuronidase enzymes. Microbes can also be genetically engineered to include a gene for a gut enzyme that provides a protective group to the xenobiotic drug in question.

### CROSS-REFERENCE TO RELATED APPLICATION

This application claims the benefit under 35 U.S.C. § 119(e)(1) to U.S. Provisional Application No. 62/626,586, filed 5 Feb. 2018, which application is incorporated herein by reference in its entirety.

### TECHNICAL FIELD

The present invention relates to methods to alter the metabolism of drugs. More particularly, the invention is directed to methods for increasing the therapeutic index of drugs by reducing toxicity, and/or increasing the efficacy associated with xenobiotic drug metabolism, using engineered microbes.

### SEQUENCE LISTING

The sequences referred to herein are listed in the Sequence Listing submitted as an ASCII text file entitled “CBI028-30_ST25.txt”—58 KB and was created on 25 Jan. 2019. The Sequence Listing entitled “CBI028-30_ST25.txt” is incorporated herein by reference in its entirety.

### BACKGROUND OF THE INVENTION

Xenobiotic metabolism refers to metabolic pathways that modify the chemical structure of xenobiotics, i.e., compounds foreign to an organism's normal biochemistry, such as food, therapeutic drugs, and poisons. These metabolic reactions often act to detoxify compounds. In particular, activated xenobiotic metabolites can be detoxified in the liver by conjugation with charged species such as glutathione (GSH), sulfate, glycine, or glucuronic acid. Sites on drugs where conjugation reactions occur include carboxyl (—COOH), hydroxyl (—OH), amino (NH₂), and sulfhydryl (—SH) groups. These reactions are catalyzed by a large group of broad-specificity transferases, such as glutathione S-transferases (GSTs) and uracil glucuronosyltransferases (UGTs).

Members of the gut microbiota throughout the human gastrointestinal (GI) tract exhibit a variety of enzymatic activities with potential impact on human health through biotransformation of detoxified xenobiotic compounds, as well as activation of prodrugs. 3-glucuronidases remove protective glucuronide groups from toxins and mutagens that have been glucuronidated in the liver and excreted into the gut with the bile. When glucuronides are deconjugated by microbial glucuronidases in the intestine, the apolar aglycone is released and can frequently be reabsorbed by the host. This can lead to high local concentrations of reactivated compounds within the gut. Furthermore, reuptake of deconjugated compounds from the gut and reglucuronidated in the liver leads to enterohepatic circulation of xenobiotic compounds, increasing their retention time in the body, thereby changing the pharmacokinetics of the drug and often leading to enhanced toxicity. Moreover, reactivation can result in GI pathologies such as damage to the gut lumen, causing inflammation, malabsorption of nutrients, nausea, and diarrhea. This is a particular problem with narrow therapeutic index (NTI) drugs that have a narrowly defined dosage range between risk and benefit. Such drugs include, but are not limited to, chemotherapeutic drugs.

For example, irinotecan, a prodrug used for cancer therapy, is metabolized to the active compound SN-38 (7-ethyl-10-hydroxy camptothecin) for treating cancer. SN-38 is detoxified in the liver by conjugation to uridine diphosphate glucuronosyltransferase 1A1 (UGT-1A1), to produce SN-38-glucuronide (SN-38G). SN-38G is actively excreted in bile. However, SN-38G can be converted back to the active compound, SN-38, by removal of the glucuronide group by the β-glucuronidase pathway found in bacteria in the GI tract. Increased concentrations of SN-38 in the intestine can cause delayed-onset diarrhea, whereas glucuronidation of SN-38 protects against this toxicity.

Methods currently being investigated for addressing reactivation of drugs include the use of antibiotics or small molecule β-glucuronidase inhibitors. However, several endogenous GI microbes are beneficial and protect against various GI disorders. Thus, killing these organisms with antibiotics is undesirable. Additionally, small molecule β-glucuronidase inhibitors are not always specific for bacterial enzymes and may have an effect on host metabolism. These inhibitors may also broadly impact the metabolism of non-target organisms and in some cases, lead to deleterious changes to the gut ecosystem.

Accordingly, additional methods for reducing adverse drug reactions due to reactivation of therapeutic drugs are highly desirable.

### SUMMARY

The present invention pertains to methods for reducing reactivation of detoxified drugs, such as narrow therapeutic index drugs. The methods include genetically engineering GI microbes in vivo or in vitro to include, for example, modifications that decrease or eliminate the presence of β-glucuronidase enzymes in the genetically modified organism, in order to prevent reactivation of the drug. Microbes can also be genetically engineered in vitro to include a gene for a liver enzyme that provides a protective group to the drug. These microbes can then be introduced to a desired subject to reduce adverse drug effects of xenobiotic agents that have been reactivated.

Accordingly, in one embodiment, a method is provided for genetically modifying a gastrointestinal (GI) microorganism. The method comprises targeting a complex that comprises a nucleic acid-targeting nucleic acid (NATNA) and a catalytically inactive site-directed protein, to a nucleic acid target region in the microorganism, wherein the target region is in proximity to a gene coding for a protein capable of reactivating a detoxified xenobiotic agent, and further wherein the complex binds to the target region to disrupt expression of the gene, thereby genetically modifying the microorganism. In certain embodiments, the catalytically inactive site-directed protein comprises a DNA binding protein, such as a protein selected from the group consisting of a catalytically inactive CRISPR-associated (Cas) protein (e.g., dCas9), a catalytically inactive zinc finger nuclease (ZFN), a zinc finger, a catalytically inactive transcription activator-like effector nuclease (TALEN), a catalytically inactive transcription activator-like effector (TALE), and a catalytically inactive meganuclease.

In additional embodiments, the NATNA comprises a guide polynucleotide such as, but not limited to, a CRISPR-Cas9 single-guide RNA (sgRNA) or a chimeric RNA/DNA guide (chRDNA).

In alternative embodiments, methods for genetically modifying a GI microorganism are provided. The methods comprise targeting a complex comprising a NATNA and a catalytically active site-directed protein to a nucleic acid target region in the microorganism, wherein the target region is in proximity to a gene coding for a protein capable of reactivating a detoxified xenobiotic agent, and further wherein the complex binds to and cleaves nucleic acid in the target region; and providing a donor polynucleotide capable of undergoing homologous recombination with the cleaved target region, whereby, upon recombination, expression of the gene is disrupted, thereby genetically modifying the microorganism. In certain embodiments, the catalytically active site-directed protein comprises a DNA binding protein, such as a protein selected from the group consisting of a Cas protein such as Cas9, a Cas9 nickase, or a Cpf1; a ZFN; a TALEN; and a meganuclease.

In additional embodiments, the NATNA comprises a CRISPR-Cas9 guide polynucleotide such as, but not limited to, a single-guide RNA (sgRNA).

In additional embodiments, the NATNA comprises a chimeric RNA/DNA (chRDNA) guide.

In certain embodiments of the methods described herein, the nucleic acid target region is in proximity to a gene encoding a β-glucuronidase.

In further embodiments of the methods described herein, the microorganism is Faecalibacterium prausnitzii or Escherichia coli.

In yet additional embodiments of the methods described herein, expression of β-glucuronidase is silenced.

In further embodiments, methods for genetically modifying a microorganism selected from the group consisting of Faecalibacterium prausnitzii and Escherichia coli are provided. The methods comprise targeting a sgRNA/dCas9 complex to a target region in proximity to a β-glucuronidase operon, wherein the complex binds to the target region to silence expression of a β-glucuronidase, thereby genetically modifying the microorganism. In certain embodiments, the microorganism is genetically modified in vivo. In other embodiments, the microorganism is genetically modified in vitro.

In further embodiments, compositions are provided. The compositions comprise a genetically modified organism produced by a method described herein, and a pharmaceutically acceptable excipient.

In additional embodiments, methods of reducing reactivation of a detoxified xenobiotic agent in a mammalian subject are provided. The methods comprise administering a composition, as described herein, or a genetically modified microorganism produced in vitro by a method as described herein, to the mammalian subject, thereby reducing reactivation of the xenobiotic agent. In certain embodiments, the mammalian subject is a human subject. In certain embodiments, the genetically modified microorganism is administered non-parenterally, such as orally.

In further embodiments, methods of reducing reactivation of a detoxified xenobiotic agent are provided. The methods comprise administering a genetically modified microorganism to the mammalian subject, wherein the genetically modified microorganism comprises a gene encoding an enzyme capable of detoxifying an active xenobiotic agent, wherein the enzyme is selected from the group consisting of a glutathione S-transferase and a uracil glucuronosyltransferase, thereby reducing reactivation of the xenobiotic agent. In certain embodiments, the genetically modified microorganism is administered non-parenterally, such as orally.

In yet further embodiments, methods for increasing the therapeutic index of a xenobiotic agent in a mammalian subject are provided. The methods comprise administering a genetically modified microorganism produced in vitro by a method as described herein to the mammalian subject, thereby increasing the therapeutic index of the xenobiotic agent. In certain embodiments, the mammalian subject is a human subject. In certain embodiments, the genetically modified microorganism is administered non-parenterally, such as orally.

In alternative embodiments, methods for increasing the therapeutic index of a xenobiotic agent in a mammalian subject are provided. The methods comprise administering a genetically modified microorganism to the mammalian subject, wherein the genetically modified microorganism comprises a gene encoding an enzyme capable of detoxifying an active xenobiotic agent, wherein the enzyme is selected from the group consisting of a glutathione S-transferase and a uracil glucuronosyltransferase, thereby increasing the therapeutic index of the xenobiotic agent. In certain embodiments, the genetically modified microorganism is administered non-parenterally, such as orally.

In any of the methods described herein, the xenobiotic agent can comprise irinotecan.

In any of the methods described herein, the xenobiotic agent can be detoxified in the liver of the mammalian subject.

These aspects and other embodiments of the present invention will readily occur to those of ordinary skill in the art in view of the disclosure herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows the effects of dCas9 complexed with specified sgRNAs on the expression of lacZ.

FIG. 2 shows the effects of dCas9 complexed with specified sgRNAs on the expression of gusA/uidA.

### DETAILED DESCRIPTION OF THE INVENTION

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a sgRNA” includes one or more sgRNAs, reference to “a mutation” includes one or more mutations, and the like.

Unless defined otherwise, all technical and scientific terms used herein have the same meaning as commonly understood by one of ordinary skill in the art to which the present invention pertains. Although other methods and materials similar, or equivalent, to those described herein can be used in the practice of the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, 2014, Cold Spring Harbor Laboratory Press, ISBN 978-1-936113-81-1; Culture of Animal Cells: A Manual of Basic Technique and Specialized Applications, 6th Edition, R. I. Freshney, 2010, Wiley-Blackwell, ISBN 978-0-470-52812-9; Transgenic Animal Technology, Third Edition: A Laboratory Handbook, 2014, C. A. Pinkert, Elsevier, ISBN 978-0124104907; The Laboratory Mouse, Second Edition, 2012, H. Hedrich, Academic Press, ISBN 978-0123820082; Manipulating the Mouse Embryo: A Laboratory Manual, 2013, R. Behringer, et al., Cold Spring Harbor Laboratory Press, ISBN 978-1936113019; PCR 2: A Practical Approach, 1995, M. J. McPherson, et al., IRL Press, ISBN 978-0199634248; Methods in Molecular Biology (Series), J. M. Walker, ISSN 1064-3745, Humana Press; RNA: A Laboratory Manual, 2010, D. C. Rio, et al., Cold Spring Harbor Laboratory Press, ISBN 978-0879698911; Methods in Enzymology (Series), Academic Press; Molecular Cloning: A Laboratory Manual (Fourth Edition), 2012, M. R. Green, et al., Cold Spring Harbor Laboratory Press, ISBN 978-1605500560; Bioconjugate Techniques, Third Edition, 2013, G. T. Hermanson, Academic Press, ISBN 978-0123822390; Methods in Plant Biochemistry and Molecular Biology, 1997, W. V. Dashek, CRC Press, ISBN 978-0849394805; Plant Cell Culture Protocols (Methods in Molecular Biology), 2012, V. M. Loyola-Vargas, et al., Humana Press, ISBN 978-1617798177; Plant Transformation Technologies, 2011, C. N. Stewart, et al., Wiley-Blackwell, ISBN 978-0813821955; Recombinant Proteins from Plants (Methods in Biotechnology), 2010, C. Cunningham, et al., Humana Press, ISBN 978-1617370212; Plant Genomics: Methods and Protocols (Methods in Molecular Biology), 2009, D. J. Somers, et al., Humana Press, ISBN 978-1588299970; Plant Biotechnology: Methods in Tissue Culture and Gene Transfer, 2008, R. Keshavachandran, et al., Orient Blackswan, ISBN 978-8173716164.

The “gastrointestinal (GI) tract,” also known as the “gut,” “digestive tract,” “digestional tract,” “GIT,” and “alimentary canal,” refers collectively to the mouth, esophagus, stomach, small intestine, large intestine, rectum, and anus.

The terms “gut microbiome,” “gut flora,” “gut microbiota,” and “GI microbiota” are used interchangeably herein and refer to the community of microorganisms that live in the GI tract of humans and other animals. Representative organisms that are part of the gut microbiome are described in detail herein.

The terms “GI microbe” and “GI microorganism” are used interchangeably herein and refer to a microorganism that can live in the gut of humans and other animals. By a GI microbe or microorganism is meant a microorganism in vivo that is present in the GI tract, or a microorganism that can live in the GI tract but that is isolated therefrom.

The terms “narrow therapeutic index (NTI)” drug and “narrow therapeutic range (NTR)” drug are used interchangeably herein and refer to drugs that have a narrowly defined dosage range between risk and benefit. These drugs are also known as “critical-dose drugs.” Such drugs can, but need not, display less than a twofold difference in the minimum toxic concentration and minimum effective concentration in the blood. For example, a drug where the ratio of the lowest concentration at which clinical toxicity occurs to the median concentration providing a therapeutic effect can be, but need not be, less than or equal to 2. NTI drugs also refer to formulations that exhibit limited or erratic absorption, formulation-dependent bioavailability, and wide intrapatient pharmacokinetic variability, such that blood-level monitoring is required. Representative classes of drugs that are considered NTI drugs are described further herein

By “raising the therapeutic index” of a drug is meant that the drug displays a higher difference in the minimum toxic concentration and minimum effective concentration in the blood as compared to the same drug as delivered under normal conditions or without the use of an engineered microbe as described herein. A high therapeutic index is preferable for a drug to have a favorable safety profile. For many drugs, there are severe toxicities that occur at sublethal doses in humans, and these toxicities often limit the maximum dose of a drug. Thus, a higher therapeutic index is preferable to a lower one because a patient would have to be administered a much higher dose of the drug to reach the toxic threshold than the dose taken to elicit the therapeutic effect.

The terms “subject,” “individual,” or “patient” are used interchangeably herein and refer to any member of the phylum Chordata, including, without limitation, mammals, such as humans and other primates, including non-human primates such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females.

The terms “effective amount” or “therapeutically effective amount” of a composition or agent, as provided herein, refer to a sufficient amount of the composition or agent to provide the desired response. In the context of the present invention, a desired amount will typically be an amount of the composition or agent that reduces adverse drug reactions to the xenobiotic drug in question, e.g., by reducing or eliminating reactivation of the drug, or by enhancing detoxification of the drug by conjugation with, for example, glutathione (GSH), sulfate, glycine, or glucuronic acid. Adverse reactions to xenobiotic drugs can include GI tract-related disorders, such as, but not limited to vomiting, diarrhea, constipation, bleeding in the GI tract, irritable bowel syndrome (IBS), diverticular disease, colon polyps, colon cancer, inflammatory bowel disease, anal fissures, anal fistulas, and the like. Other adverse reactions to the xenobiotic drug in question include systemic toxic reactions to the administered drug due to, for example, a change in pharmacokinetics of the drug in question, a change in the amount of drug circulating in the blood stream, and the like. The exact effective amount of the composition or agent required will vary from subject to subject, depending on the species, age, and general condition of the subject, the severity of the condition being treated, and the particular xenobiotic agent used, mode of administration, and the like. An appropriate “effective” amount in any individual case may be determined by one of ordinary skill in the art using routine experimentation.

“Treatment” or “treating” a particular disorder includes: (1) preventing the disorder, i.e. preventing the development of the disorder; or causing the disorder to occur with less intensity in a subject that may be predisposed to the disorder but does not yet experience or display symptoms of the disorder; (2) inhibiting the disorder, i.e., reducing the rate of development, arresting the development or reversing the disease state; and/or (3) relieving symptoms of the disorder i.e., decreasing the number of symptoms experienced by the subject.

A “disruptive modification,” as used herein, refers to a mutation or other modification that reduces or eliminates (i.e., “disrupts”) the expression or activity of a gene or protein encoded thereby. The disruptive modification may partially inactivate, fully inactivate, or delete the gene or protein. The disruptive modification may be a knockout (KO) mutation, or any modification that reduces, prevents, or blocks the biosynthesis of a product catalyzed by an enzyme encoded by the gene of interest. The disruptive modification may include, for example, a mutation in a gene encoding the enzyme, a mutation in a genetic regulatory element involved in the expression of a gene encoding the enzyme, the introduction of a nucleic acid which produces a protein that reduces or inhibits the activity of the enzyme, or the introduction of a nucleic acid or protein which inhibits the expression of the enzyme.

The terms “knockout (KO),” “silencing,” “elimination,” and “deletion” are used interchangeably herein and denote any addition or loss to a target gene sequence, or to a regulatory sequence of a cell genome so that protein expression mediated by the target gene is removed or reduced. Preferably, the target gene is rendered inoperative so that little or none of the protein product is expressed.

The terms “engineered,” “genetically engineered,” “genetically modified,” “recombinant,” “modified,” and “non-naturally occurring” indicate intentional human manipulation of the genome of an organism. Methods of genetic modification include, for example, heterologous gene expression, gene or promoter insertion or deletion, nucleic acid mutation, altered gene expression or inactivation, enzyme engineering, directed evolution, knowledge-based design, random mutagenesis methods, gene shuffling, codon optimization, and the like. Methods for genetically engineering organisms are described in detail herein.

“Gene editing” or “genome editing,” as used herein, is a type of genetic engineering and refers to the insertion, deletion, or replacement of a nucleotide sequence at a specific site in the genome of an organism or cell. Gene editing can be achieved using engineered nucleases as described herein.

A “parental microorganism” is a microorganism used to generate a genetically engineered microorganism of the present invention. The parental microorganism may be a naturally occurring microorganism (i.e., a wild-type microorganism) or a microorganism that has been previously modified (i.e., a mutant or recombinant microorganism). The microorganism may be modified to suppress expression of an enzyme that is expressed in the parental microorganism, or to express or overexpress one or more enzymes that were not expressed or overexpressed in the parental microorganism. Similarly, the microorganism of the present invention may be modified to contain one or more genes that were not contained by the parental microorganism.

Programmable nucleases enable targeted genetic modifications in a host cell genome by creating site-specific breaks at desired locations in the genome. Such nucleases include, but are not limited to, Clustered Regularly Interspaced Short Palindromic Repeats (CRISPR)-associated (Cas) nucleases, zinc-finger nucleases (ZFNs), transcription activator-like effector nucleases (TALENs), and meganucleases.

CRISPR and Cas nucleases comprise programmable adaptive immune systems of bacterial and archaeal origin. CRISPR-Cas systems are classified into two distinct classes, Class 1 and Class 2, described in detail in Koonin et al., Curr Opin Microbiol. (2017) 37:67-78; and Yan et al., Science (2019) 363:88-91. For a description of CRISPR see, e.g., Jinek et al., Science (2012) 337:816-821; PCT Publication No. WO 2013/176772, published Nov. 28, 2013; PCT Publication No. WO 2014/023828, published Feb. 19, 2015; U.S. Pat. Nos. 10,000,772; 10,113,167; 9,885,026; each of which is incorporated herein by reference in its entirety.

As used herein, “a Cas protein” such as “a Cas9 protein,” “a Cas13 protein,” “a Cas12 protein,” etc., refers to a Cas protein derived from any species, subspecies, or strain of bacteria that encodes the Cas protein of interest, as well as variants and orthologs of the particular Cas protein in question. The Cas proteins can either be directly isolated and purified from bacteria, or synthetically or recombinantly produced, or can be delivered using a construct encoding the protein including without limitation, naked DNA, plasmid DNA, a viral vector and mRNA for Cas expression. Non-limiting examples of Cas proteins include Cas9, Cas12a (Cpf1), CasM, Cas13d, CASCADE, homologs thereof, and modified versions thereof. In some embodiments, the sequence encoding the Cas protein is codon-optimized for expression in a cell of interest. In some embodiments, the Cas protein directs cleavage of one or two strands at the location of the target sequence. In some embodiments, the Cas protein lacks DNA strand cleavage activity, or acts as a nickcase. The choice of Cas protein will depend upon the particular conditions of the methods used as described herein. By a “CRISPR-Cas system,” as used herein, is meant any of the various CRISPR-Cas classes, types and subtypes.

The term “Cas9 protein,” as used herein refers to wild-type proteins derived from Class 2 Type II CRISPR-Cas9 systems, modifications of the Cas9 proteins, variants of Cas9 proteins, Cas9 orthologs, and combinations thereof. Cas9 proteins can be derived from any of various bacterial species having genomes that encode such proteins. Variants and modifications of Cas9 proteins are known in the art. U.S. Pat. Nos. 9,260,752; 9,410,198; 9,909,122; 9,725,714; 9,803,194; and 9,809,814 (each of which is incorporated herein by reference in its entirety) teach a large number of exemplary wild-type Cas9 polypeptides including the sequence of SpyCas9. Modifications and variants of Cas9 proteins are also discussed. Non-limiting examples of Cas9 proteins include Cas9 proteins from S. pyogenes (GI:15675041); Listeria innocua Clip 11262 (GI:16801805); Streptococcus mutans UA159 (GI:24379809); Streptococcus thermophilus LMD-9 (S. thermophilus A, GI:11662823; S. thermophilus B, GI:116627542); Lactobacillus buchneri NRRL B-30929 (GI:331702228); Treponema denticola ATCC 35405 (GI:42525843); Francisella novicida U112 (GI:118497352); Campylobacter jejuni subsp. Jejuni NCTC 11168 (GI:218563121); Pasteurella multocida subsp. multocida str. Pm70 (GI:218767588); Neisseria meningitidis Zs491 (GI:15602992); and Actinomyces naeslundii (GI:489880078).

By “dCas protein” is meant a nuclease-deactivated Cas protein, also termed “catalytically inactive,” “catalytically dead,” or “dead Cas protein.” Such molecules lack all or a portion of endonuclease activity and can therefore be used to regulate genes in an RNA-guided manner (Jinek et al., Science (2012) 337:816-821). This is accomplished by introducing mutations that inactivate Cas nuclease function. For Cas9, this is typically accomplished by mutating both of the two catalytic residues (D10A in the RuvC-1 domain, and H840A in the HNH domain, numbered relative to SpyCas9) of the gene encoding Cas9. It is understood that mutation of other catalytic residues to reduce activity of either or both of the nuclease domains can also be carried out by one skilled in the art. In doing so, dCas9 is unable to cleave dsDNA but retains the ability to sequence-specifically bind DNA. The Cas9 double mutant with changes at amino acid positions D10A and H840A completely inactivates both the nuclease and nickase activities. Targeting specificity is determined by complementary base-pairing of a single guide RNA to the genomic locus and the protospacer adjacent motif (PAM).

By “nCas,” as used herein, is meant a Cas nickase that maintains the ability to bind to and make a single-strand break at a target site. In the case of “nCas9,” the molecule will typically include a mutation in one, but not both of the Cas9 endonuclease domains (HNH and RuvC).

As used herein, “dual-guide RNA” refers to a two-component RNA system for a polynucleotide component capable of associating with a cognate Cas protein. A representative CRISPR Class 2 Type II CRISPR-Cas-associated dual-guide RNA includes a Cas-crRNA and Cas-tracrRNA, paired by hydrogen bonds to form secondary structure (see, e.g., Jinek et al., Science (2012) 337:816-21). A Cas-dual-guide RNA is capable of forming a nucleoprotein complex with a cognate Cas protein, wherein the complex is capable of targeting a nucleic acid target sequence complementary to the spacer sequence.

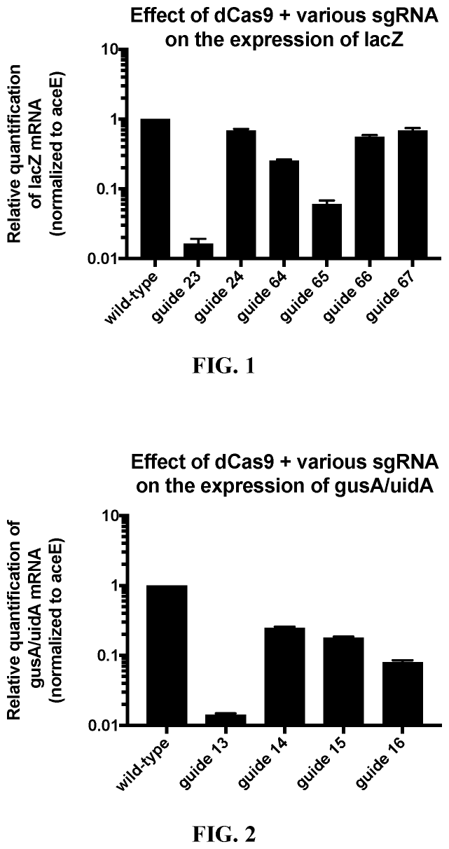

The term “sgRNA” refers to a single-guide RNA, i.e., a single, contiguous polynucleotide sequence. sgRNA interacts with a cognate Cas protein essentially as described for tracrRNA/crRNA polynucleotides. Ran et al., Nature (2015) 520:186-191 (including all extended data) present the crRNA/tracrRNA sequences and secondary structures of eight Type II CRISPR-Cas9 systems (see Extended Data FIG. 1 of Ran, et al.). Further, Fonfara, et al., Nucleic Acids Research (2014) 42:2577-2590 (including all Supplemental Data, in particular Supplemental Figure S11) present the crRNA/tracrRNA sequences and secondary structures of eight Type II CRISPR-Cas9 systems. See, also, PCT Publication No. WO 2013/176772, published Nov. 28, 2013; U.S. Pat. Nos. 10,000,772 and 10,113,167; each of which is incorporated herein in its entirety.

A Cas9 single-guide RNA (Cas9-sgRNA) is a guide RNA wherein the Cas9-crRNA is covalently joined to the Cas9-tracrRNA, often through a tetraloop, and forms a RNA polynucleotide secondary structure through base-pair hydrogen bonding. See, e.g., Jinek et al., Science (2012) 337:816-821; PCT Publication No. WO 2013/176772, published Nov. 28, 2013; U.S. Pat. Nos. 10,000,772 and 10,113,167; each of which is incorporated herein by reference in its entirety.

A “nucleic acid-targeting nucleic acid” (NATNA), also known as a “guide polynucleotide,” refers to one or more polynucleotides that guide a protein, such as a Cas nuclease, or a deactivated Cas nuclease, to preferentially target a nucleic acid target sequence present in a polynucleotide (relative to a polynucleotide that does not comprise the nucleic acid target sequence). NATNAs can comprise ribonucleotide bases (e.g., RNA); deoxyribonucleotide bases (e.g., DNA); combinations of ribonucleotide bases and deoxyribonucleotide bases (e.g., RNA/DNA chimeric molecules) such as single-guide and dual-guide RNA/DNA chimeric molecules (chRDNAs) (see, e.g., U.S. Pat. Nos. 9,580,701; 9,650,617; 9,688,972; 9,771,601; and 9,868,962 (each of which is incorporated herein by reference in its entirety)); nucleotides; nucleotide analogs; modified nucleotides; and the like; as well as synthetic, naturally occurring, and non-naturally occurring modified backbone residues or linkages. Thus, a NATNA, as used herein, site-specifically guides a protein, such as Cas9, to a target nucleic acid.

Many NATNAs are known including, but not limited to, sgRNA (including miniature and truncated sgRNAs as described in U.S. Published Patent Application No. 2017/0114334, published Apr. 27, 2017; and U.S. Published Patent Application No. 2017/0051276, published Feb. 23, 2017; each of which is incorporated herein by reference in its entirety); alternative CRISPR nucleic acid-targeting Type II nucleic acid scaffolds, including those described in e.g., U.S. Pat. Nos. 9,771,600; 9,970,029; 10,100,333; 9,816,093; 9,677,090; 9,745,562; 9,816,081; 9,957,490; 10,023,853; 10,125,354; and 10,138,472; each of which is incorporated herein by reference in its entirety; crRNA, dual-guide RNA, including but not limited to, crRNA/tracrRNA molecules; and the like; the use of which depends on the particular Cas protein. Also useful are 2-bit and 3-bit split-nexus NATNAs (sn-NATNAs), such as single-guide and dual-guide sn-Cas polynucleotides, described in e.g., U.S. Pat. Nos. 9,745,600; 9,580,727; 9,970,026; and 9,970,027; each of which is incorporated herein by reference in its entirety. For a non-limiting description of other exemplary NATNAs, see, e.g., PCT Publication No. WO 2014/150624, published Sep. 29, 2014; PCT Publication No. WO 2015/200555, published Mar. 10, 2016; PCT Publication No. WO 2016/201155, published Dec. 15, 2016; PCT Publication No. WO 2017/027423, published Feb. 16, 2017; PCT Publication No. WO 2017/070598, published Apr. 27, 2017; and PCT Publication No. WO 2016/123230, published Aug. 4, 2016; each of which is incorporated herein by reference in its entirety.

“Cpf1,” also known as “Cas12a,” refers to a CRISPR-Cas RNA-guided DNA endonuclease found in CRISPR Type V systems. The PAM for Cas12a is a “TTN” motif located 5′ to its protospacer target, as opposed to a 3′ “NGG” PAM motif used by Cas9. Cpf1 binds a crRNA that carries the protospacer sequence for base-pairing to the target. Unlike Cas9, Cpf1 does not require a separate tracrRNA and is devoid of a tracrRNA gene at the Cpf1-CRISPR locus. Thus, Cpf1 only requires a crRNA that is approximately 43 nucleotides (nt) in length, 24 nt of which are the protospacer and 19 nt the constitutive direct repeat sequence. Cpf1 appears to be directly responsible for cleaving the 43 base crRNAs apart from the primary transcript (Fonfara et al., Nature (2016) 532:517-521).

The term “CASCADE” refers to a CRISPR Type I multiprotein complex known as “CRISPR-associated complex for antiviral defense.” For a description of the CASCADE complex, see, e.g., Jore et al., Nature Structural and Molecular Biology (2011) 18:529-536. Modified CASCADE systems are described in, e.g., U.S. Pat. No. 9,885,026, incorporated herein by reference in its entirety.

As used herein, a programmable nuclease (e.g., a Cas9 protein), or a catalytically inactive programmable nuclease (e.g., a dCas9 protein) is said to “target” a polynucleotide if a NATNA/programmable nuclease complex associates with, binds, and/or cleaves (in the case of a catalytically active programmable nuclease) or binds to but does not cleave (in the case of a catalytically inactive programmable nuclease) a polynucleotide at the nucleic acid target region within the polynucleotide. In certain embodiments, the target region is “in proximity to” a gene coding for a protein, i.e., the target region can be adjacent to, operably linked to, or even within a gene of interest.

As used herein, a “site-directed polypeptide or protein” refers to a polypeptide that recognizes and/or binds to a nucleic acid target sequence or the complement of the nucleic acid target sequence. The site directed polypeptide, alone or in combination with polynucleotides such as NATNAs, will bind to a nucleic acid target sequence or to the complement of the nucleic acid target sequence.

As used herein, the term “cognate” refers to biomolecules that interact, such as a site-directed polypeptide and its NATNA; a site-directed polypeptide/NATNA complex (a nucleoprotein complex) capable of site-directed binding to a nucleic acid target sequence complementary to the NATNA binding sequence; and the like.

As used herein, the terms “complex,” “nucleoprotein complex,” “NATNA/Cas complex,” and “NATNA/dCas complex,” refer to complexes comprising a NATNA and a protein that bind to a nucleic acid target sequence. The Cas protein of the complex can effect a blunt-ended double-strand break, a double-strand break with sticky ends, nick one strand, or perform other functions on the nucleic acid target sequence.

“Transcription activation-like effectors” (TALEs) are DNA binding proteins of bacterial origin. The TAL effector DNA-binding domain recognizes specific individual base pairs in a target DNA sequence by using a known cipher involving two key amino acid residues, also referred to as the repeat variable di-residues (RVDs). See, e.g., Mussolino et al., Nucleic Acids Res. (2011) 39:9283-9293. Depending on the TALE protein sequence, TALEs can bind any DNA base (G, T, A, C). A large number of TALEs are known in the art. Several TALE DNA binding domains can be fused together and engineered to bind any contiguous DNA sequence. Typically, about 15 TALE DNA binding domains are fused together to recognize a 15 nucleotide long DNA sequence. TALEs can be fused to transcriptional activators and repressors. Engineered TALEs can be used for transcriptional activation or repression in a cell.

“Transcription activation-like effector nucleases” (TALENs) are TALEs that are fused to the DNA-cleaving domain of a restriction enzyme such as FokI. TALENs are engineered to bind and cleave any desired DNA sequence. TALENs are typically used for genome engineering of an organism. See, e.g., Mussolino et al., Nucleic Acids Res. (2011) 39:9283-9293, for a description of TALENs.

“Meganucleases” or “homing endonucleases” refer to a family of enzymes that recognize, bind, and cleave specific DNA sequences (reviewed in Stoddard, B, Mobile DNA (2014) 5:7). The DNA recognition site of meganucleases are typically 12 to 40 base pairs. A large number of meganucleases are known in the art. Meganucleases can be engineered to bind and cleave any DNA sequence. Meganucleases can also be engineered such that they are catalytically inactive and can bind but not cleave DNA. Meganucleases can be fused to other proteins such as transcriptional activators and repressors or other nucleases. Engineered meganucleases can be used for transcriptional activation or repression or genome engineering of a cell.

“Zinc fingers” are DNA binding proteins or DNA binding protein domains. The proteins or protein domains are often but not always coordinated with one or more zinc ions that recognize particular DNA sequences. A large number of zinc finger domains and proteins are known in the art (see e.g. Miller et al., EMBO J. (1985) 4:1609-1614; Rhodes et al., Sci. Amer. (1993) February: 56-65; and Kug, A., J. Mol. Biol. (1999) 293:215-218). Depending on the zinc finger sequence, one zinc finger domain typically binds a triplet of DNA bases. Several zinc fingers can be fused together and engineered to bind any target DNA sequence. Generally, about 5 zinc finger DNA binding domains are fused together to recognize a 15 nucleotide long DNA sequence. Zinc fingers can be fused to transcriptional activators and repressors. Engineered zinc fingers can be used for transcriptional activation or repression in a cell.

“Zinc finger nucleases” (ZFNs) are engineered zinc fingers that are fused with the DNA-cleaving domain of a restriction enzyme such as FokI. ZFNs can be engineered to bind and cleave any target DNA sequence. Engineered ZFNs are typically used for genome engineering of an organism. See e.g. Carroll et al., Nat. Protoc. (2006) 1:1329-1341; and U.S. Pat. Nos. 8,034,598 and 7,914,796, each of which is incorporated herein by reference in its entirety.

By “donor polynucleotide” is meant a polynucleotide that can be directed to, and inserted into a target site of interest, to modify the target nucleic acid. All or a portion of the donor polynucleotide can be inserted into the target nucleic acid. The donor polynucleotide can be used for repair of the break in the target DNA sequence resulting in the transfer of genetic information (i.e., polynucleotide sequences) from the donor at the site or in close proximity of the break in the DNA. Accordingly, new genetic information (i.e., polynucleotide sequences) may be inserted or copied at a target DNA site. The donor can be used to insert or replace polynucleotide sequences in a target sequence, for example, to introduce a polynucleotide that encodes a protein or functional RNA (e.g., siRNA), to introduce a protein tag, to modify a regulatory sequence of a gene, or to introduce a regulatory sequence to a gene (e.g. a promoter, an enhancer, an internal ribosome entry sequence, a start codon, a stop codon, a localization signal, or polyadenylation signal), to modify a nucleic acid sequence (e.g., introduce a mutation), and the like.

Targeted DNA modifications using donor polynucleotides for large changes (e.g., more than 100 base pair (bp) insertions or deletions) traditionally use donor templates that contain homology arms homologous to sequences flanking the genomic site of alteration. Each arm can vary in length, but is typically longer than about 25 bp, such as longer than 30 bp, such as 30-1500 bp, e.g., 30-1500 bp, such as 30 to 100 . . . 200 . . . 300 . . . 400 . . . 500 . . . 600 . . . 700 . . . 800 . . . 900 . . . 1000 . . . 1500 bp or any integer between these values. However, these numbers can vary, depending on the size of the donor polynucleotide and the target polynucleotide. Donor polynucleotides can be used to generate large modifications, including insertion of reporter genes such as fluorescent proteins or antibiotic-resistant markers.

For smaller insertions, single-stranded oligonucleotides containing flanking sequences on each side that are homologous to the target region can be used, and can be oriented in either the sense or antisense direction relative to the target locus. The length of each arm can vary, but the length of at least one arm is typically longer than about 10 bases, such as from 10-150 bases, e.g., 10 . . . 20 . . . 30 . . . 40 . . . 50 . . . 60 . . . 70 . . . 80 . . . 90 . . . 100 . . . 110 . . . 120 . . . 130 . . . 140 . . . 150, or any integer within these ranges. However, these numbers can vary, depending on the size of the donor polynucleotide and the target polynucleotide. In some embodiments, the length of at least one arm is 10 bases or more. In other embodiments, the length of at least one arm is 20 bases or more. In yet other embodiments, the length of at least one arm is 30 bases or more. In some embodiments, the length of at least one arm is less than 100 bases. In further embodiments, the length of at least one arm is greater than 100 bases. For single-stranded DNA oligonucleotide design, typically an oligonucleotide with up to 100-150 bp total homology is used. The mutation is introduced in the middle, giving 50-75 bp homology arms for a donor designed to be symmetrical about the target site.

A “genomic region” is a segment of a chromosome in the genome of a host cell that is present on either side of the nucleic acid target sequence site or, alternatively, also includes a portion of the nucleic acid target sequence site. The homology arms of the donor polynucleotide have sufficient homology to undergo homologous recombination with the corresponding genomic regions. In some embodiments, the homology arms of the donor polynucleotide share significant sequence homology to the genomic region immediately flanking the nucleic acid target sequence site; it is recognized that the homology arms can be designed to have sufficient homology to genomic regions farther from the nucleic acid target sequence site.

The terms “wild-type,” “naturally occurring,” and “unmodified” are used herein to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in and can be isolated from a source in nature. The wild-type form, appearance, phenotype, or strain serve as the original parent before an intentional modification. Thus, mutant, variant, chimeric, engineered, recombinant, and modified forms are not wild-type forms.

As used herein, the terms “nucleic acid,” “nucleotide sequence,” “oligonucleotide,” and “polynucleotide” are interchangeable. All refer to a polymeric form of nucleotides. The nucleotides may be deoxyribonucleotides (DNA) or ribonucleotides (RNA), or analogs thereof, and they may be of any length. Polynucleotides may perform any function and may have any secondary structure and three-dimensional structure. The terms encompass known analogs of natural nucleotides and nucleotides that are modified in the base, sugar and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include methylated nucleotides and nucleotide analogs. Nucleotide structure may be modified before or after a polymer is assembled. Following polymerization, polynucleotides may be additionally modified via, for example, conjugation with a labeling component or target-binding component. A nucleotide sequence may incorporate non-nucleotide components. The terms also encompass nucleic acids comprising modified backbone residues or linkages, that (i) are synthetic, naturally occurring, and non-naturally occurring, and (ii) have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), and morpholino structures.

Polynucleotide sequences are displayed herein in the conventional 5′ to 3′ orientation.

As used herein, the term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

As used herein, the term “sequence identity” generally refers to the percent identity of bases or amino acids determined by comparing a first polynucleotide or polypeptide to a second polynucleotide or polypeptide using algorithms having various weighting parameters. Sequence identity between two polypeptides or two polynucleotides can be determined using sequence alignment by various methods and computer programs (e.g., BLAST, CS-BLAST, FASTA, HIMMER, L-ALIGN, etc.), available through the worldwide web at sites including GENBANK (ncbi.nlm.nih.gov/genbank/) and EMBL-EBI (ebi.ac.uk.). Sequence identity between two polynucleotides or two polypeptide sequences is generally calculated using the standard default parameters of the various methods or computer programs. Generally, the various proteins for use herein will have at least about 75% or more sequence identity to the wild-type or naturally occurring sequence of the protein of interest, such as about 80%, such as about 85%, 90%, 91%, 92%, 93%, 94%, 95%, 96%, 97%, 98%, 99%, or complete identity.

As used herein, “double-strand break” (DSB) refers to both strands of a double-stranded segment of nucleic acid being severed. In some instances, if such a break occurs, one strand can be said to have a “sticky end” wherein nucleotides are exposed and not hydrogen bonded to nucleotides on the other strand. In other instances, a “blunt end” can occur wherein both strands remain fully base paired with each other.

As used herein, the term “recombination” refers to a process of exchange of genetic information between two polynucleotides.

As used herein, “nucleic acid repair” such as, but not limited to DNA repair, encompasses any process whereby cellular machinery repairs damage to a nucleic acid molecule contained in the cell. The damage repaired can include single-strand breaks, double-strand breaks, or misincorporation of bases.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like.

As used herein the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Additionally, multicistronic constructs can include multiple coding sequences which use only one promoter by including a 2A self-cleaving peptide, an IRES element, etc. Accordingly, some polynucleotide elements may be operably linked but not contiguous.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to an amino acid polymer that has been modified through, for example, acetylation, disulfide bond formation, glycosylation, lipidation, phosphorylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology (see, e.g., standard texts discussed herein). Further, essentially any polypeptide or polynucleotide can be custom ordered from commercial sources.

The term “binding” as used herein includes a non-covalent interaction between macromolecules (e.g., between a protein and a polynucleotide, between a polynucleotide and a polynucleotide, and between a protein and a protein). Such non-covalent interaction is also referred to as “associating” or “interacting” (e.g., when a first macromolecule interacts with a second macromolecule, the first macromolecule binds to second macromolecule in a non-covalent manner). Some portions of a binding interaction may be sequence-specific; however, all components of a binding interaction do not need to be sequence-specific, such as a protein's contacts with phosphate residues in a DNA backbone. Binding interactions can be characterized by a dissociation constant (Kd). “Affinity” refers to the strength of binding. An increased binding affinity is correlated with a lower Kd. An example of non-covalent binding is hydrogen bond formation between base pairs.

As used herein, the term “isolated” can refer to a nucleic acid or polypeptide that, by the hand of a human, exists apart from its native environment and is therefore not a product of nature. “Isolated” means substantially pure. An isolated nucleic acid or polypeptide can exist in a purified form and/or can exist in a non-native environment such as, for example, in a recombinant cell.

As used herein, a “host cell” generally refers to a biological cell. A cell can be the basic structural, functional and/or biological unit of a living organism. A cell can originate from any organism having one or more cells. Examples of host cells include, but are not limited to: a prokaryotic cell, eukaryotic cell, a bacterial cell, an archaeal cell, a cell of a single-cell eukaryotic organism, a protozoa cell, a cell from a plant, an algal cell, seaweeds, a fungal cell, an animal cell, a cell from an invertebrate animal, a cell from a vertebrate animal, a cell from a mammal, and the like. Further, a cell can be a stem cell or progenitor cell.

The present invention is directed to methods for genetically engineering GI microbes to aid in increasing the therapeutic index of drugs, such as narrow therapeutic index drugs, by reducing the toxicity or increasing the efficacy associated with xenobiotic drug metabolism, e.g., by reducing reactivation of detoxified drugs. The methods include genetically engineering GI microbes in vivo or in vitro to include modifications of components of a β-glucuronidase operon to decrease or eliminate the presence of β-glucuronidase enzymes in the genetically modified organism. Microbes can also be genetically engineered to include a gene for a liver enzyme that provides a protective group to the drug in question and if produced in vitro, the genetically engineered organisms can then be provided to a subject to ultimately reduce the negative effects of detoxified drugs.

As explained herein, xenobiotic drugs can be detoxified in the liver by reactions that add protective groups, such as glutathione (GSH) or glucuronic acid, to the drugs. These reactions often use broad-specificity transferases, e.g., glutathione S-transferases (GSTs) and uracil glucuronosyltransferases (UGTs). The conjugated compounds are typically much more water soluble than the respective aglycones and are often biochemically and biologically inactive. These detoxified molecules are then excreted into the gut with the bile and eliminated from the body via the gut and through the bladder into the urinary tract. However, some microorganisms found in the natural gut microbiome produce enzymes, such as β-glucuronidases, which remove the protective groups. This can lead to high local concentrations of reactivated compounds within the gut with negative, and sometimes life-threatening side-effects to the subject in question.

This is a particular problem with narrow therapeutic index (NTI) drugs, also known as “narrow therapeutic range (NTR)” drugs and “critical-dose drugs.” These drugs have a narrowly defined dosage range between risk and benefit. NTI drugs also refer to formulations that exhibit limited or erratic absorption, formulation-dependent bioavailability and wide intrapatient pharmacokinetic variability such that blood-level monitoring is required.

Representative classes of drugs that are considered NTI drugs include, without limitation, chemotherapeutic drugs, such as irinotecan, 5-fluorouracil, methotrexate, and immune-oncology effectors; antiarrythmic drugs, such as quinidine, procainamide, disopyramide, and amiodarone; levothyroxine drug products; antiepileptic drugs (AEDs), such as phenytoin, carbamazepine, lamotrigine, oxcarbazepine, and the like; warfarin compounds; and transplant drugs and anti-rejection medications. Other specific known NTI drugs include, without limitation, cyclosporine, digoxin, ethosuximide, lithium, procainamide, and theophylline. Additionally, drugs, such as nonsteroidal anti-inflammatory drugs (NSAIDs) that block COX enzymes and reduce prostaglandins throughout the body can cause significant intestinal damage through the actions of GI microbial enzymes. These drugs include, without limitation, aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin are captured under the definition of NTI drugs herein.

A number of naturally occurring gut microbes are known that play a role in xenobiotic metabolism, such as but not limited to those that produce a β-glucuronidase capable of removing glucuronic acid protective groups. Such microbes are known and described, for example, in Dabek et al., FEMS Microbiol. Ecol. (2008) 66:487-495; and Pollet et al., Structure (2017) 25:967-977. These organisms include, without limitation, bacteria in the phyla Bacteroidetes, Firmicutes, Proteobacteria, and Actinobacteria. Representative gut organisms include, without limitation, Clostridium spp., e.g., Clostridium perfringens; Roseburia spp., e.g., Roseburia hominis and Roseburia intestinalis; Ruminococcus spp. such as Ruminococcus gnavus; Faecalibacterium spp., e.g., Faecalibacterium prausnitzii; Bacteroides spp., e.g., Bacteroides ovatus; Escherichia spp., e.g., E. coli; Streptococcus spp., e.g., Streptococcus agalactiae; Eubacterium spp., such as Eubacterium eligens.

A variety of β-glucuronidase enzymes produced by organisms in the mammalian gut microbiome are known. Pollet et al., Structure (2017) 25:967-977, describes over 3000 β-glucuronidase proteins discovered using the Human Microbiome Project GI database. In members of the Enterobacteriaceae family, such as E. coli, β-glucuronidases are typically produced via the gus operon which often includes the following components: a gusR (uidR) gene that encodes a β-glucuronidase repressor; a gusA gene (uidA), that encodes β-glucuronidase; and gusB (uidB) and gusC (uidC) genes that confer glucuronide-inducible glucuronide transport activity when expressed together. See, e.g., Liang et al., J. Bacteriol. (2005) 187:2377-2385. Two homologous operons also exist in the F. prausnitzii genome sequence.

Multiple strategies for altering xenobiotic metabolism of gut bacteria in order to reduce or prevent the production of β-glucuronidase enzymes can be used in the present invention. For example, an organism can be engineered in vitro to lack the β-glucuronidase gene, or to inhibit expression of the gene, and can be delivered to outcompete the existing organism in the GI tract, thereby reducing or eliminating the metabolic pathway from the gut. Alternatively, an organism can be engineered in vitro to lack the β-glucuronidase gene or to inhibit expression of the gene, and can be delivered to the gut, and further programmed to transfer the engineered genetic material to a gut microbe of interest, such as F. prausnitzii, and/or E. coli, through a bacterial conjugation reaction. In other embodiments, the engineered genetic material is transferred to selected organisms in the gut via specific bacterial viruses (bacterial phages). In some embodiments, the phage can carry genetic material that encodes genome editing enzymes so that bacteria that are infected are modified in situ. In another embodiment, an organism can be engineered to sequester the deactivated xenobiotic agent, such as SN-38G, and the engineered microbes can be delivered to consume as much of the deactivated xenobiotic agent, such as SN-38G, as possible and then be shed from the colon, thereby expediting the removal of the drug and limiting its exposure to xenobiotic metabolism. In another example, an organism that has been engineered to convert the active xenobiotic compound, such as SN-38, back to the non-toxic compound, such as SN-38G, can be delivered to the gut and thereby counteract the xenobiotic metabolism of the GI microbe in question, such as F. prausnitzii, and/or E. coli.

Accordingly, microorganisms that normally produce β-glucuronidases, such as those described herein, can be genetically engineered to include a disruptive modification in order to modify the production of β-glucuronidase enzymes, thereby reducing or eliminating the removal of glucuronic acid protective groups from detoxified drugs. This, in turn, can reduce the incidence of undesirable side-effects of active xenobiotic agents. The disruptive modification can reduce or eliminate the expression of a β-glucuronidase gene product. For example, transcription of the gene can be partially inactivated, fully inactivated, or the gene can be eliminated. The modification can reduce, prevent, or block the biosynthesis of a β-glucuronidase. The disruptive modification may include, for example, a modification to a component of the gus operon, including for example, a mutation in a genetic regulatory element involved in the expression of a β-glucuronidase enzyme. In some cases, the disruptive modification is a knockout (KO) modification, such that the expression of β-glucuronidase is eliminated or silenced.

In some embodiments, the modification includes silencing β-glucuronidase gene expression by, for example, interfering with expression of β-glucuronidase such as by producing a barrier that prevents components of the endogenous gene transcription machinery to provide expression of the β-glucuronidase gene product. In this embodiment, any component of a gus operon, if present in the bacterium in question, can be targeted, such as any one of gusA, gusB or gusC; all three of gusA, gusB and gusC; gusA and gusB; gusA and gusC; gusB and gusC, or any combination of gusA, gusB and gusC, so long as the production of β-glucuronidase is impeded. For example, a target region can include all or a portion of the 5′ UTR as well as the start of the coding sequence, and/or can be within the coding region itself.

This can be achieved using any of several methods known in the art, such as, but not limited to, the use of a NATNA, as defined herein, that preferentially targets a nucleic acid target sequence present in a genomic region. The NATNA can be associated with a nucleic acid binding molecule, such as a DNA binding protein, that binds to, but does not cleave, the target sequence, such as a site-directed polypeptide or protein, including for example, a catalytically inactive endonuclease, such that transcription of the desired gene is blocked. The catalytically inactive endonucleases for use in this embodiment include, without limitation, those from the CRISPR-Cas systems, zinc fingers, zinc finger nucleases (ZFNs), transcription activator-like effector nucleases (TALENs), transcription activator-like effectors (TALEs), meganucleases, megaTALs, Argonautes (Ago; see, e.g., Gao et al., Nature Biotechnology (2016) 34:768-773; and U.S. Pat. No. 10,125,375, incorporated herein by reference in its entirety), and others known to one of skill in the art.

The DNA binding protein is directed to a site in proximity to a β-glucuronidase gene (or other gene of interest), or regulatory element thereof, by the NATNA, binds the site, and blocks transcription factors so that the gene product is not expressed. By “in proximity to” is meant a site near to, such as adjacent to, or operably linked to, a β-glucuronidase gene (or other gene of interest), or even a site within the gene. The NATNA and DNA binding protein can be delivered together, e.g., as a ribonucleoprotein complex, or the NATNA and binding protein can be provided separately. If the NATNA and the binding protein are delivered separately, the nucleoprotein will form a complex with the NATNA in the targeted cell and will therefore be delivered to a region in proximity of the β-glucuronidase gene to prevent transcription thereof.

Methods of designing particular NATNAs are known and described herein. See the examples herein, as well as Briner et al., Molecular Cell (2014) 56:333-339. To do so, the genomic sequence for the gene to be targeted is first identified. The exact region of the selected gene to target will depend on the specific application. For example, in order to activate or repress a target gene using, for example, Cas activators or repressors, guide polynucleotides can be targeted to the promoter driving expression of the gene of interest. For genetic knock-outs, guide polynucleotides are commonly designed to target 5′ regions of the gene because such regions are likely to be effective whether using Cas9, nCas9, or dCas9. Using the methods described herein, any desired nucleic acid sequence for modification can be targeted, including without limitation, protein coding sequences, such as β-glucuronidase genes

If CRISPR complexes are used, they can be produced using methods well known in the art. For example, guide RNA components of the complexes can be produced in vitro and Cas components can be recombinantly produced and then the two complexed together using methods known in the art. Additionally, cell lines including, but not limited to HEK293 cells, are commercially available that constitutively express Streptococcus pyogenes Cas9 as well as S. pyogenes Cas9-GFP fusions. In this instance, cells expressing Cas9 can be transfected with the guide RNA components and complexes are purified from the cells using standard purification techniques, such as but not limited to affinity, ion exchange and size exclusion chromatography. Furthermore, bacteria can be transformed with plasmids that contain the genes for expressing Cas9, sgRNA, and other components necessary for the enzymes to properly function. See, e.g., Jinek et al., Science (2012) 337:816-821.

In some embodiments, the site-directed protein is a Cas protein, such as a Cas9 protein, that has been engineered to be catalytically inactive (dCas) so that the protein binds to a nucleic acid target region in the selected bacterial genome when in association with a cognate NATNA, but does not cleave the genome. In such embodiments, the Cas protein, such as dCas9, is directed in proximity to the β-glucuronidase gene using a guide polynucleotide, such as a sgRNA or a dgRNA, in order to block the endogenous transcription machinery of the cell.

In other embodiments, β-glucuronidase gene expression is knocked out using active Cas proteins or Cpf1. For example, guide polynucleotides can be designed to target particular regions of a gene present in the target bacterial strain, and when complexed with a programmable endonuclease, the guides bring the endonuclease in proximity with the target sequence for cleavage. Guide polynucleotides are designed using methods well known in the art to target sequences upstream of a PAM sequence and preferably unique to the target compared to the rest of the genome. For example, purified guide RNAs can be generated by PCR amplification of annealed guide RNA oligos or in vitro transcription of a linearized guide RNA-containing plasmid, such as a commercially available plasmid.

Representative complexes are those between a Cas protein, such as a Cas9 protein, with a sgRNA (sgRNA/Cas9 complex). The complex can be directed precisely toward sites of interest within the host cell genome. The guide polynucleotides, such as sgRNAs, can be designed to target any DNA sequence containing the appropriate PAM necessary for each Cas endonuclease, such as Cas9. A double-strand break is then produced and insertions or deletions of nucleotides at the site of the double-strand break can be used to knock out or silence the gene of interest.

For example, a genomic engineering procedure such as lambda red recombineering, or suicide plasmid-driven homologous recombination, can be performed in order to change a particular genomic sequence from X to Y. Plasmids can be designed to express Cas9 and a guide RNA that targets X in these engineered cells, which kills all the cells that have not been successfully engineered, and spares the cells that now contain sequence Y in place of sequence X.

Alternatively, programmable endonuclease molecules can be used that retain site-directed binding capability but lack the ability to make double-strand breaks in the target sequence. For example, a Cas nickase can be used that maintains the ability to bind to and make a single-strand break at a target site. For Cas9, such a mutant (termed “nCas9” herein) will typically include a mutation in one, but not both of the Cas9 endonuclease domains (HNH and RuvC). Thus, an amino acid mutation at position D10A or H840A in Cas9, numbered relative to S. pyogenes, can result in the inactivation of the nuclease catalytic activity and convert Cas9 to a nickase enzyme that makes single-strand breaks at the target site. In this embodiment, when expressed in a cell with a guide polynucleotide, such as sgRNA designed to target the bacterial genome, the cell should not die, but one of several DNA repair pathways will be activated and result in opening the genome at the site of the ssDNA break, thus enhancing genome editing efficiency.

In certain embodiments where catalytically active endonucleases, such as Cas9 or nCas9 are used, a linear or circular DNA construct can also be provided as a donor for homologous recombination that can result in the insert of a new gene, removal of an endogenous gene, or both.

Although in some of the embodiments described herein, a Cas9-sgRNA is used as an exemplary guide polynucleotide, it will be recognized by one of skill in the art that other guide polynucleotides that site-specifically guide endonucleases, such as CRISPR-Cas proteins to a target nucleic acid can be used.

Ina representative embodiment, the β-glucuronidase-encoding gene, such as gusA, is targeted. Guide polynucleotides that target this region can be readily designed based on the known sequences of the β-glucuronidase gene in question. See, e.g., Pollet et al., Structure (2017) 25:967-977, which provides over 3000 such genes based on sequences found in the Human Microbiome Project GI database.

The gene editing system, e.g., a CRISPR-Cas system, can be delivered to a gut microbe in vivo. Typically, delivery of exogenous genetic material to a host organism in vivo occurs in the cytoplasmic compartment. Standard approaches to deliver DNA to a bacterial cell include transformation, conjugation, or transduction. Conveniently, in one embodiment of the present invention, the genetic material for use herein is cloned into a bateriophage (phage) specific for a gut microbe of interest and the genetically engineered phage can be delivered to a subject in order to transform gut microbes in vivo to turn off or reduce transcription of the β-glucuronidase gene, thereby reducing reactivation of glucuronidated xenobiotic agents. The targeted gut microbe will in part depend on the particular xenobiotic agent provided to the subject.

Phages for use herein typically include a proteinaceous capsid and tail structure which together serve to deliver genetic information to the targeted host cell. A phage capable of delivering DNA of non-phage origin to a bacterial cell is referred to as a transducing particle (IP). One of skill in the art can readily design TPs of defined DNA content such that a particular microbe can be transduced with the TP. See, e.g., Chung et al., J. Molec. Biol. (1990) 216:911-926 (1990); and Chung et al., J. Molec. Biol. (1990) 216:927-938. The process of generating a TP normally involves transferring the DNA packaging-related sequence elements of the phage to a plasmid. The phage DNA packaging machinery then recognizes the plasmid as self and loads the non-self DNA into the capsid. The TP can then be used to deliver the plasmid to a target cell population.

For example, phages specific for F. prausnitzii appear to be present in the human gut. See, e.g., Roux et al., PLoS One (2012) 7:e40418. One or more of these phages, or newly identified phages, can be isolated and used for TP production. ATP production methodology specific for F. prausnitzii, or other desired GI microbe targets, can be combined with a genome engineering technology to enable the genetic manipulation of the target microorganism in vivo.

As explained herein, the bacteria which a phage can infect and propagate within define the host range of the virus. The host range of a phage is normally limited to several strains of the same species but in some instances, can extend across the genera level. See, e.g., Gill et al., Current pharmaceutical biotech. (2010) 11:2-14. Thus, phages and TPs, can be used as highly specific DNA delivery tools due to their primarily narrow host range. TPs are thus useful for in situ bacterial engineering or killing, as phages offer high specificity and efficiencies of DNA transfer that plasmids or conjugation cannot currently meet in a complex uncontrolled environment such as the gut lumen. Methods for delivering CRISPR-Cas systems to microorganisms in vivo using phages are known, and described e.g., in Bikard et al., Nature Biotech. (2014) 32:1146-1151. Using known methods, the use of TPs to engineer any resident gut bacterial population in vivo is possible.

Another method for delivering the genetic machinery for modifying xenobiotic drug metabolism in vivo includes conjugation, which is a method for DNA transformation in bacteria. See, e.g., Lederberg et al., Science (1953) 118:169-175. The ability of bacteria to perform conjugation contributes to horizontal gene transfer and thus genome plasticity. In conjugations, plasmid DNA is transferred from one cell to another by a conjugative type IV secretion system (T4SS). See, for example, Ilangovan et al., Trends Microbiol. (2015) 23:301-310. Conjugative T4SS is a multiprotein secretion apparatus that is found in both gram-negative and gram-positive bacteria. Although the T4SS DNA transfer process in gram-negative and gram-positive organisms is similar, there are also differences that account for the differing physiology between the two main types of bacteria. The conjugation process can occur in three distinct steps. In the first and second steps, DNA is processed and recruited to the T4SS. In the third step, the processed DNA is translocated from one cell to another through the T4SS, which is one type of membrane secretion apparatus. Initiation of conjugation requires the formation of a multiprotein-DNA complex, called the relaxosome, at the origin of transfer (oriT). The enzyme relaxase with other accessory proteins, plays a crucial role in guiding the DNA through the T4SS to the recipient cell (Ilangovan et al., Trends Microbiol. (2015) 23:301-310).

In gram-positive bacteria, there are distinct conjugative transfer mechanisms, which include the ability to transfer broad-host-range plasmids such as pIP501, and the Enterococcus sex pheromone-responsive plasmid, pCF10 (Goessweiner-Mohr et al., Microbiol Spectr. (2014) 2:PLAS-0004-2013). Both plasmids mediate single-stranded DNA transfer in gram-positive bacteria. Alternatively, the conjugative transfer system found in Streptonyces mediates double-stranded DNA transfer. In a recent study, it was demonstrated that broad host-range conjugative plasmids can be delivered to diverse soil communities, which has promising applications for delivering broad host-range conjugative plasmids to other diverse communities such as the human gut microbiota. See, Klumper et al., ISME J. (2015) 9:934-945. Accordingly, as with phage delivery, conjugation reactions will also find use in delivering microbes to the gut in order to transfer genetic material to gut microorganisms in vivo.

In other embodiments of the present invention, microorganisms are genetically engineered in vitro and the genetically engineered microorganisms are administered to a subject in order to populate the GI microbiome and reduce the degree of reactivation of deactivated xenobiotic agents. As described herein, in this embodiment, microbes can be genetically engineered to produce disruptive modifications to β-glucuronidase-producing genes or to components of gus operons, as described herein. Genetically engineered organisms can also be manipulated to include genes coding for enzymes, such as glutathione S-transferases (GSTs) and uracil glucuronosyltransferases (UGTs), in order to provide glutathione and/or glucuronic acid groups to active xenobiotic agents in the intestine, such as those that have been reactivated due to β-glucuronidase enzymes present in the gut environment. Methods for genetically engineering microorganisms in vitro are well known and include, for example, CRISPR-Cas technologies as described herein. The gene encoding the desired enzyme can be incorporated as a donor polynucleotide into the target bacterial genome using homologous recombination. For example, a Cas endonuclease, such as Cas9 and guide polynucleotides that target a specific desired locus in the host cell genome, are produced.

Guide polynucleotides are designed using methods described herein. A Cas endonuclease, such as Cas9, or a variant thereof, can be purified from bacteria using bacterial Cas9 expression plasmids as described herein. For example, His-tagged Cas9 can be expressed in bacterial cells and then purified using nickel affinity chromatography. Alternatively, purified Cas9 can be purchased from a variety commercial sources, such as New England Biosciences (NEB), Ipswich, Mass.; and Thermo Fisher Scientific, Waltham, Mass.

Cas9 RNP delivery to target cells can be accomplished via lipid-mediated transfection or electroporation. Successful transformation can be confirmed, e.g., by isolating individual clones and screening the target locus using Sanger sequencing or analyzing cleavage efficiency using a restriction digest-based assay, such as a T7 endonuclease assay or a Surveyor assay. High-throughput screening techniques can also be employed, including, but not limited to, flow cytometry techniques, such as, fluorescence-activated cell sorting (FACS)-based screening platforms, microfluidics-based screening platforms, emulsion/droplet-based analysis methods, and the like. These techniques are well known in the art and reviewed in e.g., Wojcik et al., Int. J. Molec. Sci. (2015) 16:24918-24945.

Other methods for genetically engineering selected microbes include lambda red recombineering (see, e.g., Court et al., Annual Review of Genetics (2002) 36:361-388; Sawitzke et al., Methods Enzymol. (2013) 533: 157-177; Datta et al., Proc. Nat. Acad. Sci. USA, (2008) 105:1-10; and Reisch et al., Scientific Reports (2015) 5: 15096). Lambda red recombineering can also be used with CRISPR-Cas systems as described in Reisch et al., Scientific Reports (2015) 5: 15096. This system uses two plasmids and linear double-stranded DNA. One plasmid encodes a programmable endonuclease, such as a CRISPR-Cas9, another plasmid encodes a guide RNA, such as a sgRNA and the lambda RED enzymes, and the linear dsDNA contains homology to the bacterial genome and the targeted genetic change. Each plasmid and the linear DNA are transformed into the bacteria sequentially.

Other methods for genetically engineering microbes include standard recombinant DNA techniques, well known in the art.

In all of the embodiments described herein, the various components for use in the methods can be produced by synthesis, or for example, using expression cassettes encoding the site-directed protein and the NATNA. The various components can be produced recombinantly in a host cell. These components can be present on a single cassette or multiple cassettes, in the same or different constructs. Expression cassettes typically comprise regulatory sequences that are involved in one or more of the following: regulation of transcription, post-transcriptional regulation, and regulation of translation. Expression cassettes can be introduced into a wide variety of organisms including bacterial cells, yeast cells, plant cells, and mammalian cells. Expression cassettes typically comprise functional regulatory sequences corresponding to the organism(s) into which they are being introduced.

In one aspect, all or a portion of the various components for use herein are produced from vectors, including expression vectors, comprising polynucleotides encoding therefor. Vectors useful for producing components for use in the present methods include plasmids, viruses (including phage), and integratable DNA fragments (i.e., fragments integratable into the host genome by homologous recombination). A vector replicates and functions independently of the host genome, or can in some instances, integrate into the genome itself. Suitable replicating vectors will contain a replicon and control sequences derived from species compatible with the intended expression host cell. Transformed host cells are cells that have been transformed or transfected with the vectors constructed using recombinant DNA techniques.

General methods for construction of expression vectors are known in the art. Expression vectors for most host cells are commercially available. There are several commercial software products designed to facilitate selection of appropriate vectors and construction thereof, such as insect cell vectors for insect cell transformation and gene expression in insect cells, bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation, and gene expression and methods to easily enable cloning of such polynucleotides. SnapGene™ (GSL Biotech LLC, Chicago, Ill.; snapgene.com/resources/plasmid_files/your_time_is_valuable/), for example, provides an extensive list of vectors, individual vector sequences, and vector maps, as well as commercial sources for many of the vectors.

Expression cassettes typically comprise regulatory sequences that are involved in one or more of the following: regulation of transcription, post-transcriptional regulation, and regulation of translation. Expression cassettes can be introduced into a wide variety of organisms including bacterial cells, yeast cells, mammalian cells, and plant cells. Expression cassettes typically comprise functional regulatory sequences corresponding to the host cells or organism(s) into which they are being introduced. Expression vectors can also include polynucleotides encoding protein tags (e.g., poly-His tags, hemagglutinin tags, fluorescent protein tags, bioluminescent tags, nuclear localization tags). The coding sequences for such protein tags can be fused to the coding sequences or can be included in an expression cassette.

In some embodiments, polynucleotides encoding one or more of the various components are operably linked to an inducible promoter, a repressible promoter, or a constitutive promoter.

Several expression vectors have been designed for expressing NATNAs, such as guide polynucleotides. See, e.g., Shen et al., Nat. Methods (2014) 11:399-402. Additionally, vectors and expression systems are commercially available, such as from New England Biolabs (NEB; Ipswich, Mass.) and Clontech Laboratories (Mountain View, Calif.). Vectors can be designed to simultaneously express a target-specific NATNA using a U2 or U6 promoter, a Cas protein and/or a dCas protein, and, if desired, a marker protein, for monitoring transfection efficiency and/or for further enriching/isolating transfected cells by flow cytometry.

Vectors can be designed for expression of various components of the described methods in prokaryotic or eukaryotic cells. Alternatively, transcription can be in vitro, for example using T7 promoter regulatory sequences and T7 polymerase. Other RNA polymerase and promoter sequences can be used.

Vectors can be introduced into and propagated in a prokaryote. Prokaryotic vectors are well known in the art. Typically, a prokaryotic vector comprises an origin of replication suitable for the target host cell (e.g., oriC derived from Escherichia coli, pUC derived from pBR322, pSC101 derived from Salmonella, 15A origin derived from p15A, and bacterial artificial chromosomes). Vectors can include a selectable marker (e.g., genes encoding resistance for ampicillin, chloramphenicol, gentamicin, and kanamycin). Zeocin™ (Life Technologies, Grand Island, N.Y.) can be used as a selection in bacteria, fungi (including yeast), plants, and mammalian cell lines. Accordingly, vectors can be designed that carry only one drug-resistant gene for Zeocin for selection work in a number of organisms. Useful promoters are known for expression of proteins in prokaryotes, for example, T5, T7, Rhamnose (inducible), Arabinose (inducible), and PhoA (inducible). Furthermore, T7 promoters are widely used in vectors that also encode the T7 RNA polymerase. Prokaryotic vectors can also include ribosome binding sites of varying strength, and secretion signals (e.g., mal, sec, tat, ompC, and pelB). In addition, vectors can comprise RNA polymerase promoters for the expression of NATNAs. Prokaryotic RNA polymerase transcription termination sequences are also well known (e.g., transcription termination sequences from Streptococcus pyogenes).

Integrating vectors for stable transformation of prokaryotes are also known in the art (see, e.g., Heap et al., Nucleic Acids Res. (2012) 40:e59).

Expression of proteins in prokaryotes is typically carried out in E. coli with vectors containing constitutive or inducible promoters directing the expression of either fusion or non-fusion proteins.

A wide variety of RNA polymerase promoters suitable for expression of the various components are available in prokaryotes (see, e.g., Jiang, et al., Environ Microbiol. (2015) 81:2506-2514); Estrem et al., Genes Dev. (1999) 13:2134-2147).

Methods of introducing polynucleotides (e.g., an expression vector), or ribonucleoprotein particles, into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

Once produced, microbes genetically modified in vitro, and/or containing constructs including machinery to genetically engineer gut microbes in vivo, can be formulated into compositions for delivery to the GI tract of the subject to be treated. Compositions of the present invention include the constructs and one or more pharmaceutically acceptable excipients. Typically, the compositions are formulated for non-parenteral administration. For example, the compositions are formulated as oral compositions, suppositories, aerosol, intranasal, and sustained release formulations. Methods of preparing such formulations are known, or will be apparent, to those skilled in the art. See, e.g., Remington's Pharmaceutical Sciences, Mack Publishing Company, Easton, Pa.

Oral vehicles include such normally employed excipients as, for example, pharmaceutical grades of mannitol, lactose, starch, magnesium, stearate, sodium saccharin cellulose, magnesium carbonate, and the like. These oral compositions may be taken in the form of solutions, suspensions, tablets, pills, capsules, sustained release formulations, or powders, and contain from about 10% to about 95% of the active ingredient, preferably about 25% to about 70%.

For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents, such as water, aqueous saline, or other known substances, can be employed with the present invention. The nasal formulations may also contain preservatives such as, but not limited to, chlorobutanol and benzalkonium chloride. A surfactant may be present to enhance absorption of the active ingredients by the nasal mucosa.

Controlled or sustained release formulations are made by incorporating the active ingredients into carriers or vehicles such as liposomes, nonresorbable impermeable polymers such as ethylenevinyl acetate copolymers and Hytrel® copolymers (DuPont, Hayward, Calif.), swellable polymers such as hydrogels, or resorbable polymers, such as collagen and certain polyacids or polyesters, such as those used to make resorbable sutures.

A composition can also include an antimicrobial agent for preventing or deterring unwanted microbial growth. An antioxidant can also be present in the composition. Antioxidants are used to prevent oxidation, thereby preventing the deterioration of the components of the preparation.

The number of constructs in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy,” Current edition, Williams & Williams, the “Physician's Desk Reference,” Current edition, Medical Economics, Montvale, N.J., and Kibbe, A. H., Handbook of Pharmaceutical Excipients, Current edition, American Pharmaceutical Association, Washington, D.C.

The compositions herein may optionally include one or more additional agents, such as other medications used to treat a subject for the condition in question. For example, antibiotics that act on unwanted microorganisms present in the GI tract, and the like.

At least one therapeutically effective cycle of treatment with the composition will be administered to a subject. By “therapeutically effective cycle of treatment” is intended a cycle of treatment that, when administered, brings about a positive therapeutic response, i.e., the individual undergoing treatment according to the present invention exhibits a reduction in negative side-effects from the particular xenobiotic agent being administered.

In certain embodiments, multiple therapeutically effective doses of compositions will be administered. As explained herein, the compositions of the present invention are typically, although not necessarily, administered non-parenterally, such as by oral (including buccal and sublingual), rectal, nasal, pulmonary, or vaginal administration. The pharmaceutical preparation can be in the form of a liquid solution or suspension immediately prior to administration. The foregoing is meant to be exemplary as additional modes of administration are also contemplated. The pharmaceutical compositions may be administered using the same or different routes of administration in accordance with any medically acceptable method known in the art.

The actual dose to be administered will vary depending upon the age, weight, and general condition of the subject as well as the severity of the condition being treated, the judgment of the health care professional, and particular cells and compositions being administered. Therapeutically effective amounts can be determined by those skilled in the art, and will be adjusted to the particular requirements of each particular case. Generally, a therapeutically effective amount for the use of live bacteria will be measured as colony forming units (CFU). Depending on the bacterial species and the indication, 10⁷-10¹⁴ live CFU are administered, or any integer within these ranges that produces the desired effect. If phage are administered, the phage will be measured in plaque forming units (PFU), and typically 10¹⁰-10¹⁴ PFU will be administered, or any integer within these ranges that produces the desired effect.

Administration can be in a single bolus dose, or can be in two or more doses, such as one or more days apart. The amount of composition administered will depend on the potency of the specific composition, the xenobiotic agent that is being used to treat the patient, and the route of administration.

The compositions can be administered prior to, concurrent with, or subsequent to other agents. If provided at the same time as other agents, the compositions comprising the constructs can be provided in the same or in a different composition. Thus, the constructs and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising genetically modified organisms or constructs and a dose of a pharmaceutical composition comprising at least one other agent, such as a xenobiotic agent, which in combination comprises a therapeutically effective dose, according to a particular dosing regimen. Similarly, the genetically modified microorganisms or constructs and therapeutic agents can be administered in at least one therapeutic dose. Administration of the separate pharmaceutical compositions can be performed simultaneously or at different times (e.g., sequentially, in either order, on the same day, or on different days), as long as the therapeutic effect of the combination of these substances is caused in the subject undergoing therapy.

The present invention also provides a kit. In certain embodiments, the kit of the present invention comprises one or more containers comprising constructs and modified microbes as described herein, or compositions comprising the same. The containers may be unit doses, bulk packages (e.g., multi-dose packages), or subunit doses.

The kit may comprise the components in any convenient, appropriate packaging. For example, ampules with non-resilient, removable closures (e.g., sealed glass), or resilient stoppers are most conveniently used for liquid formulations. If the genetically engineered microbes or compositions are provided as a dry formulation (e.g., freeze dried or a dry powder), a vial with a resilient stopper is normally used, so that the compositions may be easily resuspended by injecting fluid through the resilient stopper.

The kit may further comprise a suitable set of instructions relating to the use of the compositions for any of the methods described herein. The instructions generally include information as to dosage, dosing schedule, and route of administration for the intended method of use. Instructions supplied in the kit can be written instructions on a label or package insert (e.g., a paper sheet included in the kit), or machine-readable instructions (e.g., instructions carried on a magnetic or optical storage disk). Instructions referred to in the kit may also be available on websites identified in the kit.

While preferred embodiments of the present invention have been shown and described herein, it will be obvious to those skilled in the art that such embodiments are provided by way of example only. From the above description and the following Examples, one skilled in the art can ascertain essential characteristics of the present invention, and without departing from the spirit and scope thereof, can make changes, substitutions, variations, and modifications of the present invention to adapt it to various usages and conditions. Such changes, substitutions, variations, and modifications are also intended to fall within the scope of the present disclosure.

### EXPERIMENTAL

Aspects of the present invention are further illustrated in the following Examples. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric. It should be understood that these Examples, while indicating some embodiments of the present invention, are given by way of illustration only.

The following Examples are not intended to limit the scope of what the inventors regard as various aspects of the present invention.

### Example 1: Production of Bacterial Strains for Use in Gene Editing

This example describes the construction of plasmids targeting endogenous genes in Escherichia coli for genome editing.

First, target nucleotide sequences in the lacZ gene (SEQ ID NOS: 22 and 23), gusA/uidA gene (SEQ ID NOS: 24 and 25), gusB (SEQ ID NOS: 47 and 48), and gusC (SEQ ID NOS: 49 and 50) in the genome of E. coli strain MG1655 were identified (SEQ ID NOS: 1-20), using techniques known in the art. See, e.g., Jinek et al., Science (2012) 337:816-821; PCT Publication No. WO 2013/176772, published Nov. 28, 2013; and U.S. Pat. Nos. 10,000,772; 10,113,167; each incorporated herein by reference in its entirety. Two plasmids were constructed, one that expressed dCas9 (SEQ ID NO: 21) under the control of the tet promoter, and another that produced cognate sgRNAs (SEQ ID NOS: 27-36) complementary to a target nucleotide sequence in E. coli MG1655 (SEQ ID NOS: 1-20) under the control of the tet promoter. Nucleotide sequences in Table 1 correspond to target nucleotide sequences identified in E. coli strain MG1655 in the lacZ gene (SEQ ID NOS: 22 and 23), gusA/uidA gene (SEQ ID NOS: 24 and 25), and the lacZ promoter (SEQ ID NO: 26).

Both plasmids were introduced simultaneously into E. coli for use in gene editing and gene silencing experiments. The sgRNA sequences used to target the target nucleotide sequences are shown in SEQ ID NOS: 27-36.

TABLE 1
Spacer sequences used to target sgRNAs to target
nucleotide sequences in lacZ and gusA/uidA

| | | Forward | Reverse |
|---|---|---|---|
| | | target | target |
| Plasmid | Target | nucleotide | nucleotide |
| Name | Gene | sequence | sequence |
| pCB3923 | lacZ | agcggataacaatt- | gtgtgaaattgtta- |
| (guide 23) | | tcacac | tccgct |
| | | (SEQ ID NO: 1) | (SEQ ID NO: 2) |
| pCB3924 | lacZ | cgtcgccaccaatc- | tatggggattggtg- |
| (guide 24) | | cccata | gcgacg |
| | | (SEQ ID NO: 3) | (SEQ ID NO: 4 |
| pCB4264 | lacZ | cgttttacaacgtc- | agtcacgacgttgt- |
| (guide 64) | | gtgact | aaaacg |
| | | (SEQ ID NO: 5 | (SEQ ID NO: 6) |
| pCB4265 | lacZ | ggccagtgaatccg- | tgattacggattca- |
| (guide 65) | | taatca | ctggcc |
| | | (SEQ ID NO: 7 | (SEQ ID NO: 8 |
| pCB4266 | lacZ | cttcttccgcgtgc- | tctgctgcacgcgg- |
| (guide 66) | | agcaga | aagaag |
| | | (SEQ ID NO: 9 | (SEQ ID NO: 10) |
| pCB4267 | lacZ | ggcacatggctgaa- | tcgatattcagcca- |
| (guide 67) | | tatcga | tgtgcc |
| | | (SEQ ID NO: 11) | (SEQ ID NO: 12) |
| pCB4272 | gusA/ | cggcctgtgggcat- | gactgaatgcccac- |
| (guide 13) | uidA | tcagtc | aggccg |
| | | (SEQ ID NO: 13) | (SEQ ID NO: 14) |
| pCB4273 | gusA/ | actgtggaattgat- | actgtggaattgat- |
| (guide 14) | uidA | cagcgt | cagcgt |
| | | (SEQ ID NO: 15) | (SEQ ID NO: 16) |
| pCB4274 | gusA/ | tatcgtgctgcgtt- | catcgaaacgcagc- |
| (guide 15) | uidA | tcgatg | acgata |
| | | SEQ ID NO: 17) | SEQ ID NO: 18) |
| pCB4275 | gusA/ | tttgaagccgatgt- | ggcgtgacatcggc- |
| (guide 16) | uidA | cacgcc | ttcaaa |
| | | (SEQ ID NO: 19) | (SEQ ID NO: 20) |

### Example 2: dCas9 Silencing of Gus Expression and Gus Enzymatic Activity in E. coli

In order to test the ability of dCas9 to suppress gus activity in E. coli produced in Example 1, dCas9-mediated altered gene expression of gus was monitored by quantitative reverse transcription polymerase chain reaction (qRT-PCR) and enzymatic plate assays as described herein. qRT-PCR was used to evaluate whether dCas9+sgRNA would block the transcription (production of mRNA) of either the lacZ or gusA genes. These genes were chosen for analysis because the expression of the lacZ gene can be easily monitored through a colorimetric enzymatic assay, and the gusA gene is responsible for generating the 0-glucuronidation enzyme in E. coli. Gene-silencing experiments were carried out with E. coli strains that contained two plasmids, one that contained the gene for dCas9 under the control of the tet promoter, and one that contained the sequence for producing sgRNA under the control of the tet promoter. The spacer sequences used to target the sgRNA to a specific oligonucleotide sequence are listed in Table 1 and described above. The inducible tet promoter was used so that the expression of dCas9 and the sgRNA were controlled by the addition of anhydrotetracycline (ATc) to the media.

### Bacterial Growth and Induction

Growth experiments were carried out in LB medium with E. coli strains expressing lacZ-specific sgRNA guides, as described in Example 1. Growth medium was M9 (Sigma-Aldrich, St. Louis, Mo.) that was supplemented with casamino acids at 0.2% final concentration (M9CA) for the E. coli strains expressing gus-specific sgRNA guides. Isopropyl-Beta-D-Thiogalactoside (IPTG) at I mM final concentration in LB was used to induce transcription from the lacZ gene. M9CA was supplemented with gluconate as a carbon source at 0.4% final concentration. Non-metabolizable p-nitro-phenyl-b-D-glucuronide (pNpG) at a 1 mM final concentration was supplemented in M9CA medium as the inducer for gus in liquid-grown cultures. M9CA agar plates contained D-glucuronic acid instead of pNpg and 5-bromo-4-chloro-3-indolyl-B-D-glucuronide (X-GcA) at 50 μg/ml final concentration. Overnight cultures were generated in 3 ml of medium supplemented with antibiotics, carbenicillin and chloramphenicol at 100 μg/ml and 34 μg/ml final concentrations. Cultures were aerated with shaking at 30° C. The next day, overnight cultures were back-diluted in 3 ml medium supplemented with antibiotics to a final OD₆₀₀ of 0.05 and were incubated at 30° C. with shaking until the cultures reached mid-log phase. Freshly-grown-cultures were used as inocula for inoculating 100 ml of growth medium. Final OD₆₀₀ was adjusted to 0.01. Cultures were grown to early mid-log phase at 30° C. with shaking and evenly divided into two flasks. ATc was added at 1 μg/ml final concentration to one of the two flasks to induce dCas9. Cultures were continuously shaken for 3 hours at 30° C. At the end of 3 hours incubation, culture turbidity was measured and the turbidity was adjusted to OD₆₀₀ of 1. 2 ml were withdrawn and immediately mixed with 4 ml of RNAprotect Bacteria Reagent™ (Qiagen, Venlo, Netherlands) according to manufacturer's instructions. After incubation for 5 minutes at room temperature, cells were harvested by centrifugation at 4,000 rpm for 10 minutes. Supernatant was removed and cell pellets were stored at −80° C. prior to RNA isolation.

RNA Isolation, cDNA Synthesis, and qRT-PCR

RNA was extracted by the Phenol-free RNA Isolation Kit™ (Amresco, Solon, Ohio) according to manufacturer's instructions as follows. Frozen cell pellets were not allowed to thaw but immediately suspended with 100 μl of TE-lysozyme solution. Lysozyme was freshly added to TE buffer (Tris-Hcl, EDTA) (Sigma-Aldrich, St. Louis, Mo.) at 1 mg per ml final concentration. The cell suspension was incubated at room temperature for 5 minutes, mixed with 300 μl of lysis buffer and vortexed. The mixture was mixed with 200 μl pure ethanol and vortexed. The mixture was loaded on a spin column and centrifuged for 1 minute at 14000 g. The column was washed with 400 μl of wash buffer three times. The spin column was centrifuged to dry for 2 minutes at 14000 g. The column-bound RNA was eluted with 50 μl elution buffer. The eluate was treated with DNase I enzyme (New England Biosciences (NEB), Ipswich, Mass.) to remove residual genomic DNA contamination. The DNase I digestion was conducted in a reaction buffer that included 10 units of DNase I and 80 units of RNasin™ (Promega, Madison, Wis.). The reaction was incubated at 37 C for 1 hour. The eluate was cleaned-up and concentrated by the GeneJET RNA Cleanup and Concentration Micro Kit™ (Thermo Fisher Scientific, Waltham, Mass.). Purity and concentration of RNA was determined by NanoDrop™ spectrometer (Thermo Fisher Scientific, Waltham, Mass.). cDNA production was carried out with iScript cDNA Synthesis Kit™ (BioRad, Hercules, Calif.) according to manufacturer's instructions. 200 ng of RNA was used as a template in cDNA reactions. cDNA reaction volumes were increased from 20 to 50 μl, and 2 μl of the product was used as a template in the following qRT-PCR reactions.

qRT-PCR reactions were carried out with Fast SYBR Green Master Mix™ (Applied Biosystems, Foster City, Calif.). The E. coli aceE was the standard to normalize gene expression. All samples were analyzed in triplicate. The averaged CT value of ace was subtracted from the average CT values of target genes, yielding the averaged differences in CT (dCT). The averaged dCT of the wild-type was subtracted from the averaged dCT of the mutant, yielding ddCT. Relative quantity (RQ) was set to 1 for wild-type. The RQ values of mutants were calculated by the formula that is power (2, ddCT). RQ values for the 5′- and 3′-ends of the target gene were plotted to examine the differences in the transcript levels.

FIGS. 1 and 2 show the effects of dCas9 complexed with specified sgRNAs on the expression of lacZ and gusA/uidA, respectively. The location of target nucleotide sequences in the lac locus for FIG. 1 is as follows: guide 23 is lac promoter at top strand; guide 24 is H991 to D998 at bottom strand; guide 64 is V10 to W17 at top strand; guide 65 is M3 to A9 at bottom strand; guide 66 is H975 to G982; guide 67 is E981 to D988. The location of guides in the gus locus for FIG. 2 is as follows: guide 13 is D16 to L23 at top strand; guide 14 is N27 to R33 at top strand; guide 15 is F114 to P120 at bottom strand; and guide 16 is R83 to A90 at top strand. The list of guide plasmids is shown in Table 1.

As shown in FIGS. 1 and 2, induction of dCas9 and specific sgRNAs against either the lacZ or gusA genes inhibited the transcription of those genes. Loss of transcription at the gusA locus also led to loss of β-glucuronidation enzymatic activity, as determined using the following enzymatic plate assay.

### Enzymatic Plate Assay

The bacteria from the experiment described above were suspended in fresh growth media and the cell suspension was spotted on M9CA agar plates that contained X-GcA (Sigma-Aldrich, St. Louis, Mo.), and +/−ATc (Sigma-Aldrich, St. Louis, Mo.). X-GlcA is a colorimetric substrate that was added to the media and resulted in cells producing a blue color if they produced an active β-glucuronidation reaction. If the gusA gene was inhibited by dCas9+sgRNA, then the β-glucuronidation reaction was blocked and cells would not produce the blue color. One set of plates contained M9CA agar with no inducer; one set of places included M9CA agar and D-glucuronic acid; and one set of plates included M9CA agar, D-glucuronic acid and ATc. Guides 13-16 were used (see Table 1). The location of the guides is provided above.

Using this system, it was shown that induction of dCas9 and specific sgRNAs against the gusA gene inhibited the transcription of the gusA gene, which led to a loss of 3-glucuronidation enzymatic activity, observed by a loss of blue color.

### Example 3: Construction of Broad-Host Range Plasmids for In Vivo Conjugation

Plasmids for in vivo conjugation of genetic material with GI tract microbes are constructed as follows. As explained herein, bacterial conjugation requires two plasmids: a mobilization plasmid carrying genes that encode for the enzymes that transfer the donor plasmid, and a donor plasmid carrying the genes to be delivered to a new organism. A mobilization plasmid for use herein is modeled after pTA-Mob, and the donor plasmid contains oriT, as well as the genes required for turning off the β-glucuronidation genes of the target gut organisms.

### Construction of Mobilization Plasmid

A broad-host range mobilization plasmid with transfer functions is created. Plasmid pTA-Mob (Strand et al., pLoS One (2014) 9:e90372), a broad-host range mobilization plasmid, is used as a template for the new construct. The mobilization plasmid, pTA-Mob, contains the following components: Gm^{r} is a gentamycin-resistant gene; rep is the pBBR1 replication protein gene; or is the pBBR1 replication origin; trfA is the replication initiation protein gene from the RK2 replicon which is not active due to lack of RK2 replication origin oriV; Tra1 and Tra2 are regions containing the tra genes necessary for the conjugative transfer of oriT-containing plasmids; parABCDE is a stabilization region encoding the gene products ParA, B, C, D, and E; and Ctl is the central control operon of RK2.

Plasmid pTA-Mob provides transfer functions for oriT-containing plasmids and can be engineered to be maintained in a variety of bacteria E. coli or other appropriate species of bacteria are used as a donor strain to carry the mobilization plasmid for conjugative delivery of oriT-containing plasmids to a variety of recipient strains including F. prausnitzii. Plasmid pTA-Mob is 52.7 kb and can be maintained in E. coli. Plasmid pTA-MOB is a derivative of a broad-host range IncP compatibility group plasmid, RK2 and its DNA sequence is available (Pansegrau et al., J. Mol Biol. (1994) 239:623-663).

Construction of a oriT-Harboring Donor Plasmid

A donor plasmid containing the required oriT sequence is constructed. The donor plasmid is engineered to contain an origin of replication, selection markers, and genes required for inactivating β-glucuronidation in specific recipient organisms.

### Conjugative Mating

Conjugative matings are performed in vitro, as described in, e.g., Strand et al., pLoS One (2014) 9:e90372. Briefly, donor and recipient strains are grown to mid-log phase. Cells are mixed and concentrated. Then, the cell mixture is spotted on an appropriate agar plate that supports the growth of both donor and recipient cells. The agar plate is incubated overnight. The next day, the cell mixture is scraped off and plated on an appropriate selective medium. Conjugative matings are confirmed by growing cells on the appropriate selective media. Selective media includes agar plates that contain antibiotics but lack a nutrient that the donor cells need to survive such as diaminopimelic acid. On this selective media, the recipient cells must receive the plasmid-containing antibiotic-resistant genes through conjugation in order to survive, but the donor cells that require diaminopimelic acid to survive, are not able to grow. Therefore, only cells that have undergone conjugative mating survive. Surviving cells are counted and tallied.

### Example 4: Isolation of E. coli from Human Donor Fecal Samples

This Example provides a description of the isolation of E. coli strains from human donor fecal samples. This method was adapted from Gerhardt, Murray, Wood, and Krieg (editors), Methods for General and Molecular Bacteriology. ASM Press, Washington D.C. (1994) p. 205.

Briefly, strains of E. coli were isolated from human donor fecal samples (obtained from The BioCollective, Denver, Colo.) using selective growth media plating techniques. Human donor fecal samples were homogenized in phosphate buffered saline (PBS, Thermo Fisher Scientific, Waltham, Mass.) and serially diluted by a factor of ten in PBS. The homogenized and diluted fecal samples were then spread on MacConkey lactose agar plates (Thermo Fisher Scientific, Waltham, Mass.) and incubated overnight at 37° C. Colonies grew on the MacConkey lactose agar plates overnight. Bacterial colonies that displayed the attributes characteristic of E. coli, such as a pink color, were picked and grown in liquid LB media (Thermo Fisher Scientific, Waltham, Mass.) overnight with shaking at 37° C. The liquid cultures of E. coli were stored at −80° C. for future use.

In order to determine the identity of the isolated bacteria, whole genome sequencing (WGS) was performed on the genomic DNA (gDNA). Briefly, genomic DNA was isolated from the bacteria using the Qiagen DNeasy™ Blood and Tissue gDNA isolation kit, according to manufacturer's instructions (Qiagen, Venlo, Netherlands). Then, genomic DNA was sequenced with the MinION™ sequencer (Oxford Nanopore Technologies, Oxford, UK) using the Ligation Sequencing Kit™ (Oxford Nanopore Technologies, Oxford, UK), following the manufacturer's protocol, and the Nextseq sequencer (Illumina, Inc. San Diego, Calif.) using the Nextera XT DNA Library Preparation Kit™ (Illumina, Inc. San Diego, Calif.), following the manufacturer's protocol.

The sequencing results were assembled into a complete bacterial genome using the Unicycler software (github.com/rrwick/Unicycler).

From the WGS data, the sequences of the 16S ribosomal RNA (rRNA), identified using the Geneious Prime™ software (Biomatters Ltd. Auckland, NZ), was compared to the NCBI BLAST database (blast.ncbi.nlm.nih.gov/Blast.cgi), which confirmed that the isolated bacteria were proteobacteria.

Next, the genome sequence of the isolated bacteria was compared against a known E. coli reference sequence using OrthoANI genome comparison software (Lee, et al., Int J Syst Evol Microbiol. (2015) 66:1100-1103; help.ezbiocloud.net/orthoani-genomic-similarity). Genomes that were greater than 98.7% similar using this software were considered to be the same species. Therefore, strains that were isolated from human donor fecal samples were determined to be E. coli when their OrthoANI score was greater than 98.7%.

### Example 5: Isolation of Other Bacteria from Human Donor Fecal Samples

This Example provides a description of the isolation of other bacteria from human donor fecal samples. This Example is adapted from the protocols known in the art. See, e.g., Kabiri et al, Can. J. Microbiol. (2013) 59:771-777; Livingston et al., J. Clin. Microbiol. (1978) 7:448-453; Fathi et al., The Open Micorbiol. J. (2016) 10:57-63; Hartemink et al., J. Microbiol. Meth. (1999) 36:181-192; Rogosa et al. J. Bacteriol. (1951) 62:132.

Strains of target bacteria are isolated from human donor fecal samples using selective growth media plating techniques. Human donor fecal samples are homogenized in PBS and serially diluted by a factor of ten in PBS. The homogenized and diluted fecal samples are then spread on selective agar plates for the specific bacteria (Table 2) and incubated for an appropriate time, at a temperature and environment conducive to the specific growth conditions of the target bacterial species. Bacterial colonies grow on the selective agar plates. Bacteria that display the attributes characteristic of the target bacterial species are picked and grown in appropriate growth media, for an appropriate time, at a temperature and environment conducive to the specific growth conditions of the target bacterial species.

TABLE 2
Selective agar plates for bacterial isolation

| Bacterial Strain | Selective Media |
|---|---|
| Escherichia coli | MacConkey Lactose Agar |
| Escherichia coli | BRILA MUG Agar |
| Bacteroides sp. | Kanamycin Bile Esculin (KBE) Agar |
| Bacteroides sp. | Bacteroides Bile Esculin (BBE) Agar |
| Lactobacillus sp. | Rogosa Agar |

In order to determine the identity of the isolated bacteria, WGS is performed on genomic DNA (gDNA) isolated from the bacteria. From the WGS data, the sequence of the 16S ribosomal RNA (rRNA) is compared to the NCBI BLAST database (blast.ncbi.nlm.nih.gov/Blast.cgi) which identifies bacterial species that have similar 16S rRNA sequences.

Next, the genome sequence of the isolated bacteria is compared against known reference sequences from bacteria identified in the 16S rRNA comparison using the OrthoANI™ genome comparison software (help.ezbiocloud.net/orthoani-genomic-similarity). Genomes that are greater than 98.7% similar using the OrthoANI software (Lee, et al., Int J Syst Evol Microbiol. (2015) 66:1100-1103; help.ezbiocloud.net/orthoani-genomic-similarity) are considered to be the same species.

### Example 6: Construction of E. coli Strains Lacking Components of the gusABC Operon Using Lambda-Red Engineering with Antibiotic Selection

This Example describes the construction of E. coli MG1655 strains lacking gusABC, gusA or gusBC. Also, described is a method for selecting for spontaneous streptomycin-resistant variants of the gusABC knockout (KO) strain adapted from Timms et al., Molec. Genetics and Genomics MGG (1992) 232: 89-96.

### Recombineering

The linear, double-stranded DNA (dsDNA) template (SEQ ID NO: 37) used for recombineering was composed of a kanamycin resistance cassette (SEQ ID NO: 38) flanked on each side by flippase recognition target (FRT) site (SEQ ID NOS: 39 and 40) and two homology regions 1 and 2 (gusABC knockout: SEQ ID NOS: 41 and 42, gusA knockout (SEQ ID NOS: 41 and 45) and gusBC knockout (SEQ ID NOS: 46 and 42) adjacent to the intended integration site. This linear DNA template was amplified by polymerase chain reaction (PCR) from a gBlock™ (Integrated DNA Technologies (IDT), Coralville, Iowa) using primers gB primer 1 (SEQ ID NO: 43) and gB primer 2 (SEQ ID NO: 44). The PCR fragment was then gel purified and concentrated by isopropanol precipitation.

Recombineering was performed using kanamycin selection in LB media essentially as described by Thomason et al., Curr. Prot. Mol. Biol. (2007) and Datsenko et al., Proc. Natl. Acad. Sci. USA (2000) 6:6640-6645. The recombineering plasmid, adapted from Datsenko et al., Proc. Natl. Acad. Sci. USA (2000) 6:6640-6645, had a temperature sensitive replication origin and conferred resistance to carbenicillin. The plasmid carried recombineering components gam, beta and exo of the lambda red recombineering system under the control of the pBAD arabinose-inducible promoter. Additionally, the recombineering plasmid contained a DNA sequence coding for a sgRNA that was not used in this experiment. The resulting recombineering plasmid was termed “pCB4275.” The recombineering-competent strain, Scb049, was created by introducing pCB4275 into E. coli MG1655 (American Type Culture Collection (ATCC) No. 700926) by electroporation. Competent cells were prepared by diluting a saturated culture 1:100 in 100 mL of LB media in a 250 mL Erlenmeyer flask and growing at 30° C. for 2.5 to 3 hours until the OD₆₀₀ was between 0.6-0.8. The culture was split between two 50 mL conical tubes and pelleted by centrifugation at 3,000 relative centrifugal force (RCF) for 15 minutes at 4° C. The supernatant was removed by aspiration. Cells were washed with 40 mL of ice cold molecular biology grade water. Cells were resuspended in 1 mL sterile ice cold solution of 20% glycerol and 1.5% mannitol in deionized (DI) water. 100 μL of cells were mixed with 1 μL of pCB4275 miniprep DNA and transferred to a pre-chilled electroporation cuvette. Electroporation was performed at 2.5 kv in 0.2 gap cuvettes (Bio-Rad, Hercules, Calif.) using the preprogrammed Ec2 setting on a MicroPulser™ Electroporator (Bio-Rad, Hercules, Calif.). Cells were recovered at 30° C. with aeration in SOC medium (super optimal broth with catabolite repression) lacking antibiotics for one hour and then plated on LB plates with 100 μg/mL carbenicillin.

Prior to the introduction of the linear dsDNA template for recombineering, the expression of the recombineering machinery was induced by diluting a saturated culture of sCB049 1:100 in 100 mL of LB medium with 100 μg/mL carbenicillin and 0.22% of arabinose. The cells were grown at 30° C. for approximately three hours prior to the introduction of 1 μg of linear DNA template (SEQ ID NO: 37) by electroporation, as described herein. Cells were recovered with aeration at 30° C. for 2 hours in SOC medium containing 100 μg/mL carbenicillin and 0.22% arabinose. After the recovery period, the cells were plated on LB plates containing 50 μg/mL kanamycin.

Putative recombinant strains were assayed for deficiency in β-glucuronidase activity as described in Example 2. Next, genomic DNA was isolated from a strain displaying reduced β-glucuronidase activity using DNeasy™ Powersoil™ Kit (Qiagen, Venlo, Netherlands), according to the manufacturer's instructions. The genotype of these strains was confirmed by whole genome sequencing (WGS) as described in Example 4.

The strain was cured of the temperature sensitive pCB4275 plasmid and the kanamycin-resistant cassette was removed using the FLP recombinase plasmid, both as described by Datsenko et al., Proc. Natl. Acad. Sci. USA (2000) 6:6640-6645. The resulting strain was termed “sCB0511.”

### Isolation of a Spontaneous Streptomycin-Resistant Variant of the Recombineered Strain

A streptomycin-resistant variant of the gusABC KO strain (sCB0511) was generated for use in in vivo mouse studies. An overnight culture of the sCB0511, grown in LB media lacking antibiotics, was diluted 1:100 in 500 mL of LB media in a 2 L culture flask and grown at 37° C. with shaking. Incubation at 42° C. was not necessary for the generation of spontaneous streptomycin-resistant mutants, but aided in the curing of the FLP recombinase plasmid, pCP20. After 2 hours, streptomycin was added to a final concentration of 300 μg/mL and the incubation was continued. After another 4 hours of incubation, two 10 mL volumes of culture were transferred to 15 mL conical tubes and the cells were pelleted at 4,000 RCF for 15 minutes. Most of the supernatant was decanted into waste and approximately 200 μL of supernatant that remained in each tube was used to resuspend the cell pellets. The resuspended cells from each tube were plated onto LB plates with 500 μg/mL streptomycin. The plates were incubated overnight at 37° C.

A streptomycin-resistant colony was picked and grown in LB media containing 500 μg/mL streptomycin. The phenotype and genotype of the final streptomycin-resistant strain (sCB0515) was confirmed as described above.

### Example 7: Assay for β-Glucoronidase Activity in Recombineered E. coli Strains

This Example describes the assessment of β-glucuronidase in strains that have been genetically modified to be deficient in β-glucuronidase activity, including strains deleted for gusABC, gusA or gusBC.

In order to assess the relative β-glucuronidase activity of engineered strains, an in vivo assay was performed similar to the method described in Adams et al., Appl. Environ. Microbiol. (1990) 56:2021-2024. Overnight cultures of the putative gusABC KO strains grown at 37° C. in LB media were diluted 1:50 into M9 minimal media supplemented with 0.4% glycerol and 0.5% cassamino acids. The diluted cultures were each split between two 14 mL disposable culture tubes (Thermo Fisher Scientific, Waltham, Mass.) with 2 mL total volume per tube. 4-Nitrophenyl β-D-glucuronide (pNPG) was added to a final concentration of 5 mM into one of the two tubes for each strain. The tubes were incubated with aeration at 37° C. overnight. To quantify p-nitrophenol (pNP) production, 1 mL of each culture was transferred to a microcentrifuge tube and cells were pelleted at 20,000 RCF for 5 minutes. 850 μL of each supernatant was transferred to disposable polystyrene cuvettes (Thermo Fisher Scientific, Waltham, Mass.). The extinction coefficient of pNPG is not at its maximum near neutral pH. Therefore, to achieve a high level of sensitivity, the pH of the supernatants was increased by adding 50 μL of 1 M sodium bicarbonate to each cuvette and mixing. The absorbance of each supernatant was measured at 405 nm in a GENESYS 50 UV-Vis™ Spectrophotometer (Thermo Fisher Scientific, Waltham, Mass.) (Table 3).

The supernatant from an E. coli MG1655 (wt) had a high level of absorbance at 405 nm only when the culture was grown in the presence of pNPG. Of the putative gusABC KO strains, isolate 1 had a relatively high level of β-glucuronidase activity, as indicated by a high absorbance at 405 nm, and was abandoned. Supernatants from Isolates 2-4, cultured in the presence of pNPG, all had reduced absorbance at 405 nm relative to the MG1655 positive control and were therefore candidates for genotypic confirmation by WGS.

E. coli MG1655 was assayed as a positive control for β-glucuronidase activity in the described assay.

TABLE 3
Assay for β-glucuronidase activity in four putative gusABC
knock out strains

| Sample | Absorbance at 405 nm |
|---|---|
| E. coli MG1655, 5 mM pNPG | 3.409 |
| gusABC::kanR isolate 1 m, No pNPG | 0.053 |
| gusABC::kanR isolate 1, 5 mM pNPG | 2.078 |
| gusABC::kanR isolate 2, No pNPG | 0.0023 |
| gusABC::kanR isolate 2, 5 mM pNPG | 0.223 |
| gusABC::kanR isolate 3, No pNPG | 0.0223 |
| gusABC::kanR isolate 3, 5 mM pNPG | 0.271 |
| gusABC::kanR isolate 4, No pNPG | 0.0065 |
| gusABC::kanR isolate 4, 5 mM pNPG | 0.26 |

### Example 8: Preparation of Bacterial Strains for Mouse Inoculum

This Example describes methods for bacterial strain preparation to be used in in vivo mouse experiments.

The bacterial strains used in this Example were generated as described in Example 6. Strains included E. coli strepR derivative of MG1655, E. coli strepR gusA KO, and E. coli strepR gusABC KO. Briefly, on day 1, overnight cultures were grown at 37° C. in LB media supplemented with 500 μg/mL streptomycin. On day 2, 200 mL cultures of each strain were grown by diluting the overnight culture 1:100 LB media with 500 μg/ml streptomycin. Cultures were grown at 37° C. with aeration to OD₆₀₀ between 0.80-0.95. Then cells were spun down and resuspended at 4° C. in 2 ml buffer (12.5% glycerol in DI water). Aliquots were prepared in cryogenic vials and cells were frozen in liquid nitrogen. The cells were stored at −80° C. until further use.

### Example 9: Cas9-Based Engineering of Human Gut Bacteria

This Example describes the preparation of Cas9 engineered gut-derived bacteria. The method is adapted from methods described in the art. See, e.g., Jiang et al., Nature Biotech. (2013) 31:223-239; Zhao et al., Microbial Cell Factories (2016) 15: 205. Human gut bacteria are isolated as described in Examples 4 and 5.

Briefly, the Cas9 protein in combination with a sgRNA and a donor DNA is used to edit bacteria without the use of selective markers such as antibiotic-resistant genes. S. pyogenes Cas9 is cloned into a single vector. The vector comprises an inducible promoter such as a tet promoter, or a cognate biological equivalent, e.g., a DNA repressor which binds a carbon source. The inducible promoter is located upstream of the Cas9 gene and provides control of Cas9 expression through addition of a small molecule such as anhydrotetracycline (ATc). Furthermore, the vector contains a DNA sequence coding for the cognate sgRNA that targets a target site in the bacterial chromosome. The vector can comprise an inducible promoter upstream of the DNA sequence coding for the sgRNA. The sgRNA contains a 19-25 nucleotide sequence identical to a unique genome location in the bacteria. This unique genomic location is referred to as the target sequence. Furthermore, the vector can comprise a tet repressor to enable repression of Cas9 and the sgRNA. The addition of ATc to the media induces both Cas9 and sgRNA expression and results in the occurrence of dsDNA breaks caused by the Cas9/sgRNA nuclease at the target site. The vector can also comprise a DNA donor sequence. The DNA donor sequence consists of two nucleotide sequences of at least 30 nucleotides in length which are identical to the chromosome sequences 5′ and 3′ adjacent to, but not including, the target. The donor can comprise a DNA sequence corresponding to the desired genome modification of about 0.001-10 kb nucleotides. The location of this DNA is between the vector-encoded 5′ and 3′ adjacent sequences and lacks the entire target sequence or a portion thereof. The co-occurrence of dsDNA breaks at the target site and a donor DNA results in stable genomic modification by homology directed repair.

The vector-comprising sequences for the Cas9 protein, sgRNA, tet promoter, tet repressor, donor sequence, and suitable antibiotic marker, is introduced into the isolated bacteria via conjugation, transformation, or phage transduction. Bacteria containing the vector are isolated by growth on selective media corresponding to the antibiotic marker. A bacterial isolate containing the vector is then propagated and plated on solid media containing an ATc for Cas9/sgRNA expression. Bacterial survivors and Cas9/sgRNA induction is screened for editing by PCR. Engineered bacteria are then prepared for inoculation into mice as described in Example 8.

### Example 10: Effects of Engineered Bacteria in a Mouse Model of Xenobiotic Metabolism

This Example provides a method to evaluate the effects of engineered bacteria in a mouse model of xenobiotic metabolism. The protocol for generating the mouse model was adapted from Wallace et al., Chem. and Biol. (2015) 22:1238-1249).

On day one and everyday thereafter for 10 days, female Balb/cJ mice (The Jackson Laboratory, Farmington, Conn.) were administered irinotecan (50 mg/kg) (Selleckchem, Houston, Tex.) via intraperitoneal (IP) injection once daily for the length of the experiment. On day one and every day thereafter for 10 days, mice were also administered streptomycin (5 mg/ml) (Sigma-Aldrich, St. Louis, Mo.) ad libidum via their drinking water in order to kill proteobacteria in the mouse GI tract. The engineered bacteria from Example 8 were all resistant to streptomycin so that they could inhabit the mouse GI tract during the experiment.

On day two, 0.1 ml of engineered bacteria in PBS at a concentration of 10¹⁰ colony forming units per mL were administered to the mice once via oral gavage.

On days 3, 7, 8, 9, and 10, the presence of the bacteria in the mouse GI tract was confirmed by collecting fecal samples from the mice, and analyzing the fecal samples for the presence of engineered bacteria. The fecal samples were homogenized in PBS, and inoculated onto agar plates containing streptomycin (500 μg/ml). These agar plates inhibited the growth of other bacteria, and only allowed the engineered bacteria to grow. Colonies were quantified and tallied to determine the amount of bacteria that were alive in the mouse GI tract.

On days 6 through 10, the mice were observed for the occurrence of diarrhea. Diarrhea indicated whether the xenobiotic metabolism of the irinotecan caused GI toxicity.

Nine of the ten mice that did not receive engineered bacteria experienced GI toxicity from the irinotecan and displayed weight loss, diarrhea, and death. All of the mice that received engineered bacteria to prevent xenobiotic metabolism did not display diarrhea, indicating that they did not experience toxicity from xenobiotic metabolism (Table 4).

TABLE 4
Results of engineered bacteria on mouse model

| | Mice with diarrhea/total |
|---|---|
| Test Condition | mice in test group |
| Untreated | 0/10 |
| Irinotecan | 9/10 |
| Engineered bacteria | 0/10 |

Although preferred embodiments of the subject methods have been described in some detail in the Examples, it is understood that obvious variations can be made without departing from the spirit and the scope of the methods as defined by the appended claims.

1. A method for genetically modifying a gastrointestinal (GI) microorganism, comprising:
targeting a complex comprising a nucleic acid-targeting nucleic acid (NATNA) guide and a catalytically inactive Cas9 (dCas9) protein to a nucleic acid target region in the GI microorganism, wherein the nucleic acid target region is in proximity to a gene coding for a protein capable of reactivating a detoxified xenobiotic agent, and further wherein the complex binds to the nucleic acid target region to disrupt expression of the gene, thereby genetically modifying the GI microorganism.

2. (canceled)

3. (canceled)

4. (canceled)

5. (canceled)

6. The method of claim 1, wherein the NATNA guide-comprises a single-guide RNA (sgRNA).

7. (canceled)

8. A method for genetically modifying a gastrointestinal (GI) microorganism, comprising:
targeting a complex comprising a nucleic acid-targeting nucleic acid (NATNA) guide and a catalytically active Cas9 protein to a nucleic acid target region in the GI microorganism, wherein the nucleic acid target region is in proximity to a gene coding for a protein capable of reactivating a detoxified xenobiotic agent, and further wherein the complex binds to and cleaves the nucleic acid-target region; and
providing a donor polynucleotide capable of undergoing homologous recombination with the cleaved nucleic acid target region, whereby, upon recombination, expression of the gene is disrupted, thereby genetically modifying the GI microorganism.

9. (canceled)

10. (canceled)

11. (canceled)

12. The method of claim 8, wherein the Cas9 protein comprises nCas9.

13. (canceled)

14. The method of claim 8, wherein the NATNA guide comprises a single guide RNA (sgRNA).

15. (canceled)

16. The method of claim 8, wherein the nucleic acid target region is in proximity to a gene encoding a β-glucuronidase.

17. The method of claim 16, wherein the microorganism is Faecalibacterium prausnitzii.

18. The method of claim 16, wherein the microorganism is Escherichia coli.

19. The method of claim 16, whereby expression of the β-glucuronidase is silenced.

20. (canceled)

21. (canceled)

22. (canceled)

23. The method of claim 8, wherein the microorganism is genetically modified in vitro.

24. (canceled)

25. (canceled)

26. (canceled)

27. (canceled)

28. (canceled)

29. (canceled)

30. A method for increasing the therapeutic index of a xenobiotic agent in a mammalian subject, comprising:
administering a genetically modified microorganism to the mammalian subject, wherein the genetically modified microorganism comprises a gene encoding an enzyme capable of detoxifying an active xenobiotic agent, thereby increasing the therapeutic index of the xenobiotic agent.

31. The method of claim 30, wherein the genetically modified microorganism is administered non-parenterally.

32. The method of claim 31, wherein the genetically modified microorganism is administered orally.

33. The method of claim 30, wherein the xenobiotic agent comprises irinotecan.

34. (canceled)

35. The method of claim 30, wherein the mammalian subject is a human subject.

36. The method of claim 30, wherein the enzyme is selected from the group consisting of a glutathione S-transferase and an uracil glucuronosyltransferase.

37. The method of claim 30, wherein the microorganism is Faecalibacterium prausnitzii.

38. The method of claim 30, wherein the microorganism is Escherichia coli.


SEQUENCE LISTING (50 sequences)

SEQ ID NO: 1 - DNA, 20 bp, Artificial Sequence
  Synthetic Oligo for generating guide 23 sgRNA
agcggataacaatttcacac

SEQ ID NO: 2 - DNA, 20 bp, Artificial Sequence
  Synthetic Oligo complementary to SeqID NO1
gtgtgaaattgttatccgct

SEQ ID NO: 3 - DNA, 20 bp, Artificial Sequence
  Synthetic Oligo for generating guide 24 sgRNA
cgtcgccaccaatccccata

SEQ ID NO: 4 - DNA, 20 bp, Artificial Sequence
  Synthetic Oligo complementary to SeqID NO3
tatggggattggtggcgacg

SEQ ID NO: 5 - DNA, 20 bp, Artificial Sequence
  Synthetic Oligo for generating guide 64 sgRNA
cgttttacaacgtcgtgact

SEQ ID NO: 6 - DNA, 20 bp, Artificial Sequence
  Synthetic Oligo complementary to SeqID NO5
agtcacgacgttgtaaaacg

SEQ ID NO: 7 - DNA, 20 bp, Artificial Sequence
  Synthetic Oligo for generating guide 65 sgRNA
ggccagtgaatccgtaatca

SEQ ID NO: 8 - DNA, 20 bp, Artificial Sequence
  Synthetic Oligo complementary to SeqID NO7
tgattacggattcactggcc

SEQ ID NO: 9 - DNA, 20 bp, Artificial Sequence
  Synthetic Oligo for generating guide 66 sgRNA
cttcttccgcgtgcagcaga

SEQ ID NO: 10 - DNA, 20 bp, Artificial Sequence
  Synthetic Oligo complementary to SeqID NO9
tctgctgcacgcggaagaag

SEQ ID NO: 11 - DNA, 20 bp, Artificial Sequence
  Synthetic Oligo for generating guide 67 sgRNA
ggcacatggctgaatatcga

SEQ ID NO: 12 - DNA, 20 bp, Artificial Sequence
  Synthetic Oligo complementary to SeqID NO11
tcgatattcagccatgtgcc

SEQ ID NO: 13 - DNA, 20 bp, Artificial Sequence
  Synthetic Oligo for generating guide 13 sgRNA
cggcctgtgggcattcagtc

SEQ ID NO: 14 - DNA, 20 bp, Artificial Sequence
  Synthetic Oligo complementary to SeqID NO13
gactgaatgcccacaggccg

SEQ ID NO: 15 - DNA, 20 bp, Artificial Sequence
  Synthetic Oligo for generating guide 14 sgRNA
actgtggaattgatcagcgt

SEQ ID NO: 16 - DNA, 20 bp, Artificial Sequence
  Synthetic Oligo complementary to SeqID NO15
actgtggaattgatcagcgt

SEQ ID NO: 17 - DNA, 20 bp, Artificial Sequence
  Synthetic Oligo for generating guide 15 sgRNA
tatcgtgctgcgtttcgatg

SEQ ID NO: 18 - DNA, 20 bp, Artificial Sequence
  Synthetic Oligo complementary to SeqID NO17
catcgaaacgcagcacgata

SEQ ID NO: 19 - DNA, 20 bp, Artificial Sequence
  Synthetic Oligo for generating guide 16 sgRNA
tttgaagccgatgtcacgcc

SEQ ID NO: 20 - DNA, 20 bp, Artificial Sequence
  Synthetic Oligo complementary to SeqID NO19
ggcgtgacatcggcttcaaa

SEQ ID NO: 21 - protein, 1368 aa, Artificial Sequence
  Synthetic dCas9 protein sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - DNA, 3075 bp, Escherichia coli
  misc_feature (1)..(3075): lacZ DNA sequence
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 23 - protein, 1024 aa, Escherichia coli
  misc_feature (1)..(1024): lacZ protein sequence
MTMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 24 - DNA, 1812 bp, Escherichia coli
  misc_feature (1)..(1812): gusA DNA sequence
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 25 - protein, 603 aa, Escherichia coli
  misc_feature (1)..(603): gusA protein sequence
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 26 - DNA, 122 bp, Escherichia coli
  misc_feature (1)..(122): LacZ promoter
gcgcaacgcaattaatgtgagttagctcactcattaggcaccccaggctttacactttatgcttccggctcgtatgttgtgtggaattgtgagcggataacaatttcacacaggaaacagct

SEQ ID NO: 27 - DNA, 102 bp, Artificial Sequence
  Synthetic Guide 23 sgRNA DNA sequence
agcggataacaatttcacacgttttagagctagaaatagcaagttaaaataaggctagtccgttatcaacttgaaaaagtggcaccgagtcggtgctttttt

SEQ ID NO: 28 - DNA, 102 bp, Artificial Sequence
  Synthetic Guide 24 sgRNA DNA sequence
cgtcgccaccaatccccatagttttagagctagaaatagcaagttaaaataaggctagtccgttatcaacttgaaaaagtggcaccgagtcggtgctttttt

SEQ ID NO: 29 - DNA, 102 bp, Artificial Sequence
  Synthetic Guide 64 sgRNA DNA sequence
cgttttacaacgtcgtgactgttttagagctagaaatagcaagttaaaataaggctagtccgttatcaacttgaaaaagtggcaccgagtcggtgctttttt

SEQ ID NO: 30 - DNA, 102 bp, Artificial Sequence
  Synthetic Guide 65 sgRNA DNA sequence
ggccagtgaatccgtaatcagttttagagctagaaatagcaagttaaaataaggctagtccgttatcaacttgaaaaagtggcaccgagtcggtgctttttt

SEQ ID NO: 31 - DNA, 102 bp, Artificial Sequence
  Synthetic Guide 66 sgRNA DNA sequence
cttcttccgcgtgcagcagagttttagagctagaaatagcaagttaaaataaggctagtccgttatcaacttgaaaaagtggcaccgagtcggtgctttttt

SEQ ID NO: 32 - DNA, 102 bp, Artificial Sequence
  Synthetic Guide 67 sgRNA DNA sequence
ggcacatggctgaatatcgagttttagagctagaaatagcaagttaaaataaggctagtccgttatcaacttgaaaaagtggcaccgagtcggtgctttttt

SEQ ID NO: 33 - DNA, 102 bp, Artificial Sequence
  Synthetic Guide 13 sgRNA DNA sequence
cggcctgtgggcattcagtcgttttagagctagaaatagcaagttaaaataaggctagtccgttatcaacttgaaaaagtggcaccgagtcggtgctttttt

SEQ ID NO: 34 - DNA, 102 bp, Artificial Sequence
  Synthetic Guide 14 sgRNA DNA sequence
actgtggaattgatcagcgtgttttagagctagaaatagcaagttaaaataaggctagtccgttatcaacttgaaaaagtggcaccgagtcggtgctttttt

SEQ ID NO: 35 - DNA, 102 bp, Artificial Sequence
  Synthetic Guide 15 sgRNA DNA sequence
agccgggcaattgctgtgccgttttagagctagaaatagcaagttaaaataaggctagtccgttatcaacttgaaaaagtggcaccgagtcggtgctttttt

SEQ ID NO: 36 - DNA, 102 bp, Artificial Sequence
  Synthetic Guide 16 sgRNA DNA sequence
tatcgtgctgcgtttcgatggttttagagctagaaatagcaagttaaaataaggctagtccgttatcaacttgaaaaagtggcaccgagtcggtgctttttt

SEQ ID NO: 37 - DNA, 1359 bp, Artificial Sequence
  Synthetic dsDNA template for gusABC KO
ttatttccatttctcttccatgggtttctcacagataactgtgtgcaacacagaattggttaactaatcagattaaaggttgaccagtattattatcttaatgaggagtcccttatgttagaagttcctatactttctagagaataggaacttcggaataggaacttcaagatccccttagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctaaagcgctctgaagttcctatactttctagagaataggaacttcggaataggaactaagtaaaaaataacgccggagagaaaaatctccggcgtttcagattgttgacaaagtgcgcgttttttatgccggatgcggcgtaaacgccttatccagcctacaaaaactcataaattc

SEQ ID NO: 38 - DNA, 1005 bp, Artificial Sequence
  Synthetic kanamycin resistance cassette
aagatccccttagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 39 - DNA, 48 bp, Artificial Sequence
  Synthetic FRT site 1
gaagttcctatactttctagagaataggaacttcggaataggaacttc

SEQ ID NO: 40 - DNA, 46 bp, Artificial Sequence
  Synthetic FRT site 2
gaagttcctatactttctagagaataggaacttcggaataggaact

SEQ ID NO: 41 - DNA, 120 bp, Artificial Sequence
  Synthetic homology region 1 GusA
ttatttccatttctcttccatgggtttctcacagataactgtgtgcaacacagaattggttaactaatcagattaaaggttgaccagtattattatcttaatgaggagtcccttatgtta

SEQ ID NO: 42 - DNA, 120 bp, Artificial Sequence
  Synthetic homology region 2 GusC
caatgaatcaacaactctcctggcgcaccatcgtcggctacagcctcggtgacgtcgccaataacttcgccttcgcaatgggggcgctcttcctgttgagttactacaccgacgtcgctg

SEQ ID NO: 43 - DNA, 22 bp, Artificial Sequence
  Synthetic gB primer 1
ttatttccatttctcttccatg

SEQ ID NO: 44 - DNA, 22 bp, Artificial Sequence
  Synthetic gB primer 2
gaatttatgagtttttgtaggc

SEQ ID NO: 45 - DNA, 120 bp, Artificial Sequence; identical to SEQ ID NO: 42
  Synthetic homology region 1 GusA

SEQ ID NO: 46 - DNA, 120 bp, Artificial Sequence
  Synthetic homology region 1 GusB
taacaagaaagggatcttcactcgcgaccgcaaaccgaagtcggcggcttttctgctgcaaaaacgctggactggcatgaacttcggtgaaaaaccgcagcagggaggcaaacaatgaat

SEQ ID NO: 47 - DNA, 1374 bp, Escherichia coli
  misc_feature (1)..(1374): GusB DNA sequence
atgaatcaacaactctcctggcgcaccatcgtcggctacagcctcggtgacgtcgccaataacttcgccttcgcaatgggggcgctcttcctgttgagttactacaccgacgtcgctggcgtcggtgccgctgcggcgggcaccatgctgttactggtgcgggtattcgatgccttcgccgacgtctttgccggacgagtggtggacagtgtgaatacccgctggggaaaattccgcccgtttttactcttcggtactgcgccgttaatgatcttcagcgtgctggtattctgggtgctgaccgactggagccatggtagcaaagtggtgtatgcatatttgacctacatgggcctcgggctttgctacagcctggtgaatattccttatggttcacttgctaccgcgatgacccaacaaccacaatcccgcgcccgtctgggcgcggctcgtgggattgccgcttcattgacctttgtctgcctggcatttctgataggaccgagcattaagaactccagcccggaagagatggtgtcggtataccatttctggacaattgtgctggcgattgccggaatggtgctttacttcatctgcttcaaatcgacgcgtgagaatgtggtacgtatcgttgcgcagccgtcattgaatatcagtctgcaaaccctgaaacggaatcgcccgctgtttatgttgtgcatcggtgcgctgtgtgtgctgatttcgacctttgcggtcagcgcctcgtcgttgttctacgtgcgctatgtgttaaatgataccgggctgttcactgtgctggtactggtgcaaaacctggttggtactgtggcatcggcaccgctggtgccggggatggtcgcgaggatcggtaaaaagaataccttcctgattggcgctttgctgggaacctgcggttatctgctgttcttctgggtttccgtctggtcactgccggtggcgttggttgcgttggccatcgcttcaattggtcagggcgttaccatgaccgtgatgtgggcgctggaagctgataccgtagaatacggtgaatacctgaccggcgtgcgaattgaagggctcacctattcactattctcatttacccgtaaatgcggtcaggcaatcggaggttcaattcctgcctttattttggggttaagcggatatatcgccaatcaggtgcaaacgccggaagttattatgggcatccgcacatcaattgccttagtaccttgcggatttatgctactggcattcgttattatctggttttatccgctcacggataaaaaattcaaagaaatcgtggttgaaattgataatcgtaaaaaagtgcagcagcaattaatcagcgatatcactaattaa

SEQ ID NO: 48 - protein, 457 aa, Escherichia coli
  misc_feature (1)..(457): GusB amino acid sequence
MNQQLSWRTIVGYSLGDVANNFAFAMGALFLLSYYTDVAGVGAAAAGTMLLLVRVFDAFADVFAGRVVDSVNTRWGKFRPFLLFGTAPLMIFSVLVFWVLTDWSHGSKVVYAYLTYMGLGLCYSLVNIPYGSLATAMTQQPQSRARLGAARGIAASLTFVCLAFLIGPSIKNSSPEEMVSVYHFWTIVLAIAGMVLYFICFKSTRENVVRIVAQPSLNISLQTLKRNRPLFMLCIGALCVLISTFAVSASSLFYVRYVLNDTGLFTVLVLVQNLVGTVASAPLVPGMVARIGKKNTFLIGALLGTCGYLLFFWVSVWSLPVALVALAIASIGQGVTMTVMWALEADTVEYGEYLTGVRIEGLTYSLFSFTRKCGQAIGGSIPAFILGLSGYIANQVQTPEVIMGIRTSIALVPCGFMLLAFVIIWFYPLTDKKFKEIVVEIDNRKKVQQQLISDITN

SEQ ID NO: 49 - DNA, 1266 bp, Escherichia coli
  misc_feature (1)..(1266): GusC DNA sequence
atgagaaaaatagtggccatggccgttatttgcctgacggctgcctctggccttacctctgcttatgcggcgcaactggctgacgatgaagcgggactacgcatcagactgaaaaacgaattgcgcagggcggataagcccagtgctggcgcgggaagagatatttacgcatgggtacagggaggattgctcgatttcaatagtggttattattccaatattattggcgttgaaggcggggcgtattatgtttataaattaggtgctcgtgctgatatgagtacccggtggtatcttgatggtgataaaagttttggctttgccctgggggcagtaaaaataaaacccagtgaaaatagcctgcttaaattaggtcgcttcgggacggattatagttatggtagcttaccttatcgtattccgttaatggctggcagttcgcaacgtacattaccgacagtttctgaaggagcattaggttattgggctttaacaccaaatattgatctgtggggaatgtggcgttcacgagtatttttatggactgattcaacaaccggtattcgtgatgaaggggtgtataacagccagacgggaaaatacgataaacatcgcgcacgttcttttttagccgccagttggcatgatgataccagtcgctattctctgggggcatcggtacagaaagatgtttccaatcagatacaaagtattctcgagaaaagcataccgctcgacccgaattatacgttgaaaggggagttgctcggcttttacgcgcagctcgaaggtttaagtcgtaataccagccagcccaatgaaacggcgttggttagtggacaattgacctggaatgcgccgtggggaagtgtatttggcagtggtggttatttgcgccatgcaatgaatggtgccgtggtggataccgacattggctatcccttttcattaagtcttgatcgtaaccgtgaaggaatgcagtcctggcaattgggcgtcaactatcgtttaacgccgcaatttacgctgacatttgcaccgattgtgactcgcggctatgaatccagtaaacgagatgtgcggattgaaggcacgggtatcttaggtggtatgaactatcgggtcagcgaagggccgttacaagggatgaatttctttcttgctgccgataaagggcgggaaaagcgcgatggcagtacgctgggcgatcgcctgaattactgggatgtgaaaatgagtattcagtatgactttatgctgaagtaa

SEQ ID NO: 50 - protein, 421 aa, Escherichia coli
  misc_feature (1)..(421): GusC amino acid sequence
MRKIVAMAVICLTAASGLTSAYAAQLADDEAGLRIRLKNELRRADKPSAGAGRDIYAWVQGGLLDFNSGYYSNIIGVEGGAYYVYKLGARADMSTRWYLDGDKSFGFALGAVKIKPSENSLLKLGRFGTDYSYGSLPYRIPLMAGSSQRTLPTVSEGALGYWALTPNIDLWGMWRSRVFLWTDSTTGIRDEGVYNSQTGKYDKHRARSFLAASWHDDTSRYSLGASVQKDVSNQIQSILEKSIPLDPNYTLKGELLGFYAQLEGLSRNTSQPNETALVSGQLTWNAPWGSVFGSGGYLRHAMNGAVVDTDIGYPFSLSLDRNREGMQSWQLGVNYRLTPQFTLTFAPIVTRGYESSKRDVRIEGTGILGGMNYRVSEGPLQGMNFFLAADKGREKRDGSTLGDRLNYWDVKMSIQYDFMLK